(19) United States
(12) Patent Application Publication
(10) Pub. No.: US 2024/0051930 A1
(43) Pub. Date: Feb. 15, 2024

(54) HETEROAROMATIC SPERMIDINE ANALOGUES AND THEIR ANTICANCER ACTIVITY

(71) Applicant: FLORATEK PHARMA S.A., Aubonne (CH)

(72) Inventor: Dan Florin Stoicescu, Aubonne (CH)

(73) Assignee: FLORATEK PHARMA S.A., Aubonne (CH)

(21) Appl. No.: 18/275,850

(22) PCT Filed: Feb. 8, 2022
(86) PCT No.: PCT/EP2022/053045

§ 371 Date: Aug. 4, 2023

(30) Foreign Application Priority Data
Feb. 8, 2021 (GB) 2101728.0

(51) Int. Cl. C07D 311/30; A61P 35/00

(52) U.S. Cl. CPC C07D 311/30; A61P 35/00

(57) ABSTRACT

The present invention relates to chromen-4-one derivatives, and to associated multi-salts, solvates, prodrugs and pharmaceutical compositions. The present invention also relates to the use of such compounds and compositions in the treatment and prevention of cancer.

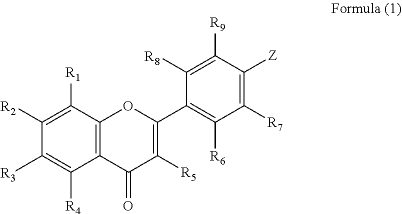

### FIELD OF THE INVENTION

The invention relates to compounds, pharmaceutical compositions comprising the same, and methods of treatment employing the same. In particular, the compounds are useful for the treatment or prevention of cancer.

### BACKGROUND

Dual-targeting or multi-targeting of malignant pathways by a single drug molecule represents an efficient, logical and alternative approach to drug combinations. A new generation of dual or multi-targeting drugs is emerging, where a single chemical entity can act on multiple molecular targets [Raghavendra N M, et al. Dual or multi-targeting inhibitors: The next generation anticancer agents. Eur J Med Chem. 2018 Jan. 1; 143:1277-1300]. The present invention uses a rational, bioinformatics and poly-pharmacological approach to design multi-target anticancer agents by combining flavonoid-like structures with a variety of polyamine chains from the spermidine class. Both broad molecule structures have been shown to target multiple cellular pathways leading to cancer inhibition [Kikuchi H, Yuan B, Hu X, Okazaki M. Chemopreventive and anticancer activity of flavonoids and its possibility for clinical use by combining with conventional chemotherapeutic agents. Am J Cancer Res. 2019; 9(8):1517-1535; Carl W. Porter, Raymond J. Bergeron and Neal J. Stolowich. Biological Properties of N⁴-Spermidine Derivatives and Their Potential in Anticancer Chemotherapy. Cancer Res. 1982 (42) (10) 4072-4078].

The role of flavonoids as potential cancer therapies includes the inhibition of activation of pro-carcinogens, inhibition of proliferation of cancer cells, selective death of cancer cells by apoptosis, inhibition of metastasis and angiogenesis, activation of immune response against cancer cells, modulation of the inflammatory cascade and the modulation of drug resistance [Kikuchi H, Yuan B, Hu X, Okazaki M. Chemopreventive and anticancer activity of flavonoids and its possibility for clinical use by combining with conventional chemotherapeutic agents. Am J Cancer Res. 2019; 9(8):1517-1535]. In spite of their promising potential in controlling the malignant process, natural flavonoids present major limitations to their clinical use due to low bioavailability and their perceived lack of specificity. Such versatile biological activity implies a great underlying complexity in the true mechanisms of action of different flavonoids, often dependent on a fine balance between pro- and anti-oxidant properties or between other beneficial and detrimental effects [Martinez Perez, et al. 2014. Novel flavonoids as anti-cancer agents: mechanisms of action and promise for their potential application in breast cancer. Biochemical Society Transactions, vol. 42, no. 4, pp. 1017-1023.]. As such, a systematic study of the structure activity relationship of the flavonoid structures is necessary and can be addressed by the rationale design of a series of molecules.

The present invention addresses some of the limitations of flavonoid derivatives by employing a novel approach of combining such moieties with a synthetic polyamine chain with multiple potential advantages: (i) multi-targeting cancer pathways (ii) inhibition of the natural polyamine pathway which is often dysregulated in cancer and (iii) facilitating transport through the cell membrane and targeting to specific intracellular structures.

It is well-known that polyamines interact with aspartate, glutamate, and aromatic residues of a given receptor and/or enzyme mainly through the formation of ion bonds, since at physiological pH, protonation of amino groups is nearly complete. From this, the hypothesis arises that a polyamine may be a universal template able to recognize different receptor systems. This hypothesis suggests that both affinity and selectivity may be fine-tuned by inserting appropriate substituents onto the amine functions and by varying the methylene chain lengths between them on the polyamine backbone [Minarini, A., Milelli, A., Tumiatti, V. et al. Synthetic polyamines: an overview of their multiple biological activities. Amino Acids 38, 383-392 (2010).]

Polyamine metabolism is often dysregulated in cancers. In addition, the polyamine pathway is a downstream target for many oncogenes [Shantz L M, Levin V A. Regulation of ornithine decarboxylase during oncogenic transformation: mechanisms and therapeutic potential. Amino Acids. 2007; 33(2):213-23]. Polyamine biosynthesis is activated in tumors, and these metabolites are important for developmental and compensatory growth in response to systemic stimuli like hormones (growth hormones, corticosteroids, androgens, and estrogens). As a result, various strategies targeting polyamine biosynthetic enzymes have been brought to the preclinical and clinical arena [Arruabarrena-Aristorena A, et al. Oil for the cancer engine: The cross-talk between oncogenic signaling and polyamine metabolism. Sci Adv. 2018; 4(1):1-1].

The most successful and widely used inhibitor of polyamine biosynthesis is 2-difluoromethylornithine (DFMO). DFMO was specifically designed to be an enzyme-activated irreversible inhibitor of ODC [Metcalf B W, et al. Catalytic irreversible inhibition of mammalian ornithine decarboxylase (E.C4.1.1.17) by substrate and product analogues. J. Am. Chem. Soc. 1978; 100:2551-2553.]

These encouraging results in selective cancers, both in vitro and in vivo led to clinical trials with DFMO as a single agent. Although DFMO was exceedingly well tolerated, there were no significant clinical responses observed in the early trials. More recently, a resurgence of interest in DFMO as a single agent has occurred in the treatment of neuroblastoma [Bassiri H, et al. Translational development of difluoromethylornithine (DFMO) for the treatment of neuroblastoma. Transl. Pediatr. 2015; 4:226-238] and as a chemoprevention agent, alone or in various combinations [Gerner E W, Meyskens F L Jr. Polyamines and cancer: old molecules, new understanding. Nat Rev Cancer. 2004 October; 4(10):781-92].

Another rational for linking polyamines to bioactive moieties is not only to use the polyamine transport system to enter the cell, but also to direct the agent to its intracellular molecular target, which can be mitochondria or other anionic structures [Murray-Stewart T R, et al. Targeting polyamine metabolism for cancer therapy and prevention. Biochem J. 2016; 473(19):2937-2953].

### SUMMARY OF THE INVENTION

A first aspect of the invention provides a compound of formula (1):

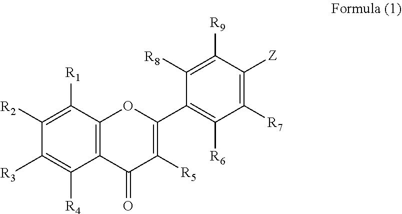

wherein:
Z is selected from:
   —NR¹¹R¹²;
   —N(R¹⁰)—(CH₂)ₚ—NR¹¹R¹²;
   —N(R¹⁰)—(CH₂)_{q}—N(R¹⁰)—(CH₂)_{q}—NR¹¹R¹²; and
   —N(R¹⁰)—(CH₂)ᵣ—N(R¹⁰)—(CH₂)ᵣ—N(R¹⁰)—(CH₂)ᵣ—NR¹¹R¹²;
R¹, R², and R⁴ independently, are selected from —OH, —O—C₁₋₄ alkyl, —OC(O)R₁₃, —OC(O)NHR¹³, —OC(O)N(R¹³)₂; or R¹ and R² together form —O—CH₂—O—;
R³, R⁴, R⁶, R⁷, R⁸, and R⁹, independently, are selected from H;
   halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R;
each —R^{β} is independently selected from a C₁-C₆ alkyl, C₂-C₆ alkenyl, C₂-C₆ alkynyl, —O(C₁₋₂ alkyl) or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
each R¹⁰ is independently selected from H, C₁₋₆ alkyl, C₂-C₆ alkenyl, C₂₋₆ alkynyl, C₃₋₁₀ cycloalkyl, and benzyl, wherein each R¹⁰, when not H, is independently optionally substituted with 1 or 2 —R^{β};
R¹¹ and R¹² are independently selected from H, C₁₋₆-alkyl, C₂-C₆ alkenyl, C₂₋₆ alkynyl, C₃₋₁₀ cycloalkyl, and benzyl, wherein each R¹¹ and R¹², when is not H, are independently optionally substituted with 1 or 2 —R^{β}; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl or benzyl;
each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl;
each p is independently an integer selected from 1 to 4;
each q is independently an integer selected from 1 to 4; and
each r is independently an integer selected from 1 to 4.

In one embodiment, the compound may be a compound of Formula (1A):

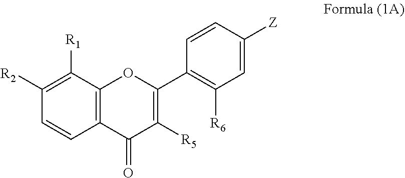

wherein R¹, R², R⁵, R⁶, and Z are as defined herein.

A second aspect of the invention provides a compound selected from the group consisting of the compounds of Table A herein.

A third aspect of the invention provides pharmaceutically acceptable salt, multi-salt, solvate or prodrug of the compound of the first or second aspect of the invention.

A fourth aspect of the invention provides a pharmaceutical composition comprising a compound of the first or second aspect of the invention, or a pharmaceutically acceptable multi-salt, solvate or prodrug of the third aspect of the invention, and a pharmaceutically acceptable excipient.

A fifth aspect of the invention provides a compound of the first or second aspect of the invention, or a pharmaceutically acceptable multi-salt, solvate or prodrug of the third aspect of the invention, or a pharmaceutical composition of the fourth aspect of the invention, for use in medicine, and/or for use in the treatment or prevention of a disease, disorder or condition. In one embodiment, the disease, disorder or condition is cancer.

A sixth aspect of the invention provides the use of a compound of the first or second aspect, a pharmaceutically effective multi-salt, solvate or prodrug of the third aspect, or a pharmaceutical composition according to the fourth aspect, in the manufacture of a medicament for the treatment or prevention of a disease, disorder or condition. Typically the treatment or prevention comprises the administration of the compound, multi-salt, solvate, prodrug or pharmaceutical composition to a subject. In one embodiment, the disease, disorder or condition is cancer.

A seventh aspect of the invention provides a method of treatment or prevention of a disease, disorder or condition, the method comprising the step of administering an effective amount of a compound of the first or second aspect, or a pharmaceutically acceptable multi-salt, solvate or prodrug of the third aspect, or a pharmaceutical composition of the fourth aspect, to thereby treat or prevent the disease, disorder or condition. Typically the administration is to a subject in need thereof. In one embodiment, the disease, disorder or condition is cancer.

### Definitions

In the context of the present specification, a “hydrocarbyl” substituent group or a hydrocarbyl moiety in a substituent group only includes carbon and hydrogen atoms but, unless stated otherwise, does not include any heteroatoms, such as N, O or S, in its carbon skeleton. A hydrocarbyl group/moiety may be saturated or unsaturated (including aromatic), and may be straight-chained or branched, or be or include cyclic groups wherein, unless stated otherwise, the cyclic group does not include any heteroatoms, such as N, O or S, in its carbon skeleton. Examples of hydrocarbyl groups include alkyl, alkenyl, alkynyl, cycloalkyl, cycloalkenyl and aryl groups/moieties and combinations of all of these groups/moieties. Typically a hydrocarbyl group is a C₁-C₁₂ hydrocarbyl group. More typically a hydrocarbyl group is a C₁-C₁₀ hydrocarbyl group. A “hydrocarbylene” group is similarly defined as a divalent hydrocarbyl group.

An “alkyl” substituent group or an alkyl moiety in a substituent group may be linear or branched. Examples of alkyl groups/moieties include methyl, ethyl, n-propyl, i-propyl, n-butyl, i-butyl, t-butyl and n-pentyl groups/moieties. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group.

An “alkenyl” substituent group or an alkenyl moiety in a substituent group refers to an unsaturated alkyl group or moiety having one or more carbon-carbon double bonds. Examples of alkenyl groups/moieties include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups/moieties. Unless stated otherwise, the term “alkenyl” does not include “cycloalkenyl”. Typically an alkenyl group is a C₂-C₁₂ alkenyl group. More typically an alkenyl group is a C₂-C₆ alkenyl group. An “alkenylene” group is similarly defined as a divalent alkenyl group.

An “alkynyl” substituent group or an alkynyl moiety in a substituent group refers to an unsaturated alkyl group or moiety having one or more carbon-carbon triple bonds. Examples of alkynyl groups/moieties include ethynyl, propargyl, but-1-ynyl and but-2-ynyl. Typically an alkynyl group is a C₂-C₁₂ alkynyl group. More typically an alkynyl group is a C₂-C₆ alkynyl group. An “alkynylene” group is similarly defined as a divalent alkynyl group.

A “haloalkyl” substituent group or haloalkyl group in a substituent group refers to an alkyl, alkenyl, or alkynyl substituent group or moiety including one or more carbon atoms and one or more halo atoms, e.g. Cl, Br, I, or F. Each halo atom replaces a hydrogen of the alkyl, alkenyl, or alkynyl substituent group or moiety. Examples include —CH₂F—CHF₂, —CHI₂, —CHBr₂, —CHCl₂, —CF₃, —CH₂CF₃ and CF₂CH₃.

An “alkoxy” substituent group or alkoxy group in a substituent group refers to an alkyl, alkenyl, or alkynyl substituent group or moiety including one or more carbon atoms and one or more oxygen atoms. Each oxygen atom replaces a carbon atom (for example the terminal or bonding carbon) of the alkyl, alkenyl, or alkynyl substituent group or moiety. Examples include —OCH₃, —OCH₂CH₃, —OCH₂CH₂CH₃, and —OCH(CH₃)(CH₃).

An “alkylthio” substituent group or alkylthio group in a substituent group refers to an alkyl, alkenyl, or alkynyl substituent group or moiety including one or more carbon atoms and one or more sulphur atoms. Each sulphur atom replaces a carbon atom (for example the terminal or bonding carbon) of the alkyl, alkenyl, or alkynyl substituent group or moiety. Examples include —SCH₃, —SCH₂CH₃, —SCH₂CH₂CH₃, and —SCH(CH₃)(CH₃).

An “alkylsulfinyl” substituent group or alkylsulfinyl group in a substituent group refers to an alkyl, alkenyl, or alkynyl substituent group or moiety including one or more carbon atoms and one or more sulfinyl groups (—S(═O)—). Each sulfinyl group replaces a carbon atom (for example the terminal or bonding carbon) of the alkyl, alkenyl, or alkynyl substituent group or moiety. Examples include —S(═O)CH₃, —S(═O)CH₂CH₃, —S(═O)CH₂CH₂CH₃, and —S(═O)CH(CH₃)(CH₃).

An “alkylsulfonyl” substituent group or alkylsulfonyl group in a substituent group refers to an alkyl, alkenyl, or alkynyl substituent group or moiety including one or more carbon atoms and one or more sulfonyl groups (—SO₂—). Each sulfonyl group replaces a carbon atom (for example the terminal or bonding carbon) of the alkyl, alkenyl, or alkynyl substituent group or moiety. Examples include —SO₂(CH₃), —SO₂(CH₂CH₃), —SO₂(CH₂CH₂CH₃), and —SO₂(CH(CH₃)(CH₃)).

An “arylsulfonyl” substituent group or arylsulfonyl group in a substituent group refers to an aryl substituent group or moiety including one or more carbon atoms and one or more sulfonyl groups (—SO₂—). Each sulfonyl group replaces a carbon atom (for example the terminal or bonding carbon) of the alkyl, alkenyl, or alkynyl substituent group or moiety. Examples include —SO₂(CH₃), —SO₂(CH₂CH₃), —SO₂(CH₂CH₂CH₃), and —SO₂(CH(CH₃)(CH₃)).

A “cyclic” substituent group or a cyclic moiety in a substituent group refers to any hydrocarbyl ring, wherein the hydrocarbyl ring may be saturated or unsaturated and may include one or more heteroatoms, e.g. N, O or S, in its carbon skeleton. Examples of cyclic groups include aliphatic cyclic, cycloalkyl, cycloalkenyl, heterocyclic, aryl and heteroaryl groups as discussed below. A cyclic group may be monocyclic, bicyclic (e.g. bridged, fused or spiro), or polycyclic. Typically, a cyclic group is a 3- to 12-membered cyclic group, which means it contains from 3 to 12 ring atoms. More typically, a cyclic group is a 3- to 7-membered monocyclic group, which means it contains from 3 to 7 ring atoms.

A “heterocyclic” substituent group or a heterocyclic moiety in a substituent group refers to a cyclic group or moiety including one or more carbon atoms and one or more heteroatoms, e.g. N, O or S, in the ring structure. Examples of heterocyclic groups include heteroaryl groups as discussed below and non-aromatic heterocyclic groups such as azetidinyl, azetinyl, tetrahydrofuranyl, pyrrolidinyl, tetrahydrothiophenyl, tetrahydropyranyl, piperidinyl, piperazinyl, morpholinyl and thiomorpholinyl groups.

An “aliphatic cyclic” substituent group or aliphatic cyclic moiety in a substituent group refers to a hydrocarbyl cyclic group or moiety that is not aromatic. The aliphatic cyclic group may be saturated or unsaturated and may include one or more heteroatoms, e.g. N, O or S, in its carbon skeleton. Examples include cyclopropyl, cyclohexyl and morpholinyl. Unless stated otherwise, an aliphatic cyclic substituent group or moiety may include monocyclic, bicyclic or polycyclic hydrocarbyl rings.

A “cycloalkyl” substituent group or a cycloalkyl moiety in a substituent group refers to a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl substituent group or moiety may include monocyclic, bicyclic or polycyclic hydrocarbyl rings.

A “cycloalkenyl” substituent group or a cycloalkenyl moiety in a substituent group refers to a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl. Unless stated otherwise, a cycloalkenyl substituent group or moiety may include monocyclic, bicyclic or polycyclic hydrocarbyl rings.

An “aryl” substituent group or an aryl moiety in a substituent group refers to an aromatic hydrocarbyl ring. The term “aryl” includes monocyclic aromatic hydrocarbons and polycyclic fused ring aromatic hydrocarbons wherein all of the fused ring systems (excluding any ring systems which are part of or formed by optional substituents) are aromatic. Examples of aryl groups/moieties include phenyl, naphthyl, anthracenyl and phenanthrenyl. Unless stated otherwise, the term “aryl” does not include “heteroaryl”.

A “heteroaryl” substituent group or a heteroaryl moiety in a substituent group refers to an aromatic heterocyclic group or moiety. The term “heteroaryl” includes monocyclic aromatic heterocycles and polycyclic fused ring aromatic heterocycles wherein all of the fused ring systems (excluding any ring systems which are part of or formed by optional substituents) are aromatic. Examples of heteroaryl groups/moieties include the following:

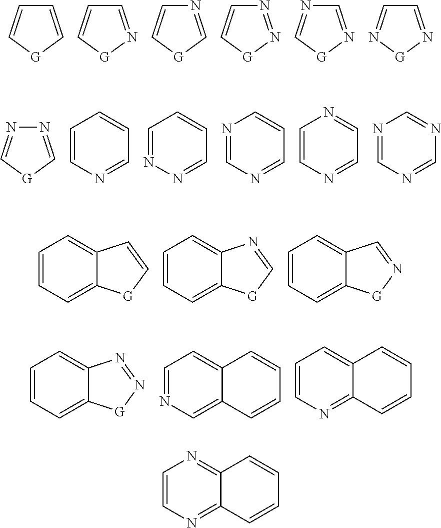

wherein G=O, S or NH.

For the purposes of the present specification, where a combination of moieties is referred to as one group, for example, arylalkyl, arylalkenyl, arylalkynyl, alkylaryl, alkenylaryl or alkynylaryl, the last mentioned moiety contains the atom by which the group is attached to the rest of the molecule. An example of an arylalkyl group is benzyl.

Typically a substituted group comprises 1, 2, 3 or 4 substituents, more typically 1, 2 or 3 substituents, more typically 1 or 2 substituents, and even more typically 1 substituent.

Unless stated otherwise, any divalent bridging substituent (e.g. —O—, —S—, —NH—, —N(R^{β})— or —R^{α}—) of an optionally substituted group or moiety must only be attached to the specified group or moiety and may not be attached to a second group or moiety, even if the second group or moiety can itself be optionally substituted.

The term “halo” includes fluoro, chloro, bromo and iodo.

Where reference is made to a carbon atom of a group being replaced by an N, O or S atom, what is intended is that:

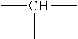

is replaced by

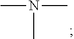

—CH₂— is replaced by —NH—, —O— or —S—;
—CH₃ is replaced by —NH₂, —OH, or —SH;
—CH═ is replaced by —N═;
CH₂═ is replaced by NH═, O═ or S═; or
CH≡ is replaced by N≡.

In the context of the present specification, unless otherwise stated, a Cₓ-C_{y} group is defined as a group containing from x to y carbon atoms. For example, a C₁-C₄ alkyl group is defined as an alkyl group containing from 1 to 4 carbon atoms. Optional substituents and moieties are not taken into account when calculating the total number of carbon atoms in the parent group substituted with the optional substituents and/or containing the optional moieties. For the avoidance of doubt, replacement heteroatoms, e.g. N, O or S, are counted as carbon atoms when calculating the number of carbon atoms in a Cₓ-C_{y} group. For example, a morpholinyl group is to be considered a C₆ heterocyclic group, not a C₄ heterocyclic group.

A “protecting group” refers to a grouping of atoms that when attached to a reactive functional group (e.g. OH) in a compound masks, reduces or prevents reactivity of the functional group.

In the context of the present specification, ═ is a double bond; ≡ is a triple bond.

The protection and deprotection of functional groups is described in ‘Protective Groups in Organic Synthesis’, 2^{nd} edition, T. W. Greene and P. G. M Wuts, Wiley-Interscience.

### DETAILED DESCRIPTION OF THE INVENTION

A first aspect of the invention provides a compound of formula (1):

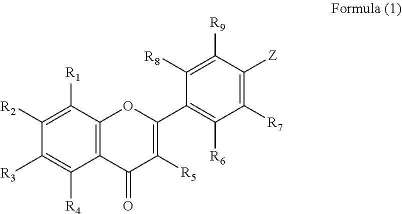

wherein:
Z is selected from:
   —NR¹¹R¹²;
   —N(R¹⁰)—(CH₂)ₚ—NR¹¹R¹²;
   —N(R¹⁰)—(CH₂)_{q}—N(R¹⁰)—(CH₂)_{q}—NR¹¹R¹²; and
   —N(R¹⁰)—(CH₂)ᵣ—N(R¹⁰)—(CH₂)ᵣ—N(R¹)—(CH₂)ᵣ—NR¹¹R¹²;
R¹, R², and R⁴ independently, are selected from —OH, —O—C₁₋₄ alkyl, —OC(O)R¹₃, —OC(O)NHR¹³, —OC(O)N(R¹³)₂; or R¹ and R² together form —O—CH₂—O—;
R³, R⁴, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β};
each —R^{β} is independently selected from a C₁-C₆ alkyl, C₂-C₆ alkenyl, C₂-C₆ alkynyl, —O(C₁₋₂ alkyl) or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
each R¹⁰ is independently selected from H, C₁₋₆ alkyl, C₂-C₆ alkenyl, C₂₋₆ alkynyl, C₃₋₁₀ cycloalkyl, and benzyl, wherein each R¹⁰, when not H, is independently optionally substituted with 1 or 2 —R^{β};
R¹¹ and R¹² are independently selected from H, C₁₋₆-alkyl, C₂-C₆ alkenyl, C₂₋₆ alkynyl, C₃₋₁₀ cycloalkyl, and benzyl, wherein each R¹¹ and R¹², when is not H, are independently optionally substituted with 1 or 2 —R^{β}; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl or benzyl;
each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl;
each p is independently an integer selected from 1 to 4;
each q is independently an integer selected from 1 to 4; and
each r is independently an integer selected from 1 to 4.
   R¹, R², and R⁵, independently, are selected from —OH, —O—C₁₋₄ alkyl, —OC(O)R₁₃, —OC(O)NHR¹³, —OC(O)N(R¹³)₂. In one embodiment, R¹, R², and R⁵, independently, are selected from —OH, and —O—C₁₋₄ alkyl. In one embodiment, R¹, R², and R⁵, independently, are selected from —OH and —OCH₃.

In one embodiment, R¹ and R² together form —O—CH₂—O—.
R³, R⁴, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. In one embodiment, R³, R⁴, R⁶, R⁷, R⁸, and R⁹, independently, are selected from H;
halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOR^{β}; and benzyl optionally substituted with 1-3 —R^{β}. In one embodiment, R³, R⁴, R⁶, R⁷, R⁸, and R⁹, independently, are selected from H;
halo; —CN; —NO₂; —R^{β}; —OH; —OR^{β}; —NH₂; —NHR^{β}; —N(RD)₂; —CHO; —COR^{β}; —COOH; —COOR^{β}; and —OCOR^{β}. In one embodiment, R³, R⁴, R⁶, R⁷, R⁸, and R⁹, independently, are selected from H;
halo; —CN; —NO₂; —R^{β}; —NH₂; —NHR^{β}; —N(R^{β})₂; —CHO; —COR^{β}; —COOH; —COOR^{β}; and —OCOR^{β}. In one embodiment, R³, R⁴, R⁶, R⁷, R⁸, and R⁹, independently, are selected from H; halo; —CN; —NO₂; —R^{β}; —NH₂; —NHR^{β}; —N(R^{β})₂; —CHO; —COR^{β}; —COOH; and —COOR^{β}. In one embodiment, R³, R⁴, R⁶, R⁷, R⁸, and R⁹, independently, are selected from H; halo; —CN; —NO₂; and —NH₂. In one embodiment, R³, R⁴, R⁶, R⁷, R⁸, and R⁹, are H.

In one embodiment, R¹, R², and R⁵, independently, are selected from —OH, —O—C₁₋₄ alkyl, —OC(O)R₁₃, —OC(O)NHR¹³, —OC(O)N(R¹³)₂; and R³, R⁴, R⁶, R⁷, R⁸, and R⁹, independently, are selected from H;
halo; —CN; —NO₂; —R; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}.

In one embodiment, R¹, R², and R⁵, independently, are selected from —OH, and —O—C₁₋₄ alkyl. For example, R³, R⁴, R⁶, R⁷, R⁸, and R⁹, are independently selected from H; halo; —CN; —NO₂; —R^{β}; —OH; —OR^{β}; —NH₂; —NHR^{β}; —N(R^{β})₂; —CHO; —COR^{β}; —COOH; —COOR^{β}; and —OCOR^{β}.

In one embodiment, R¹, R², and R⁵, independently, are selected from —OH, and —O—C₁₋₄ alkyl. For example, R³, R⁴, R⁶, R⁷, R⁸, and R⁹, are independently selected from H; halo; —CN; —NO₂; —R^{β}; —NH₂; —NHR^{β}; —N(R^{β})₂; —CHO; —COR^{β}; —COOH; —COOR^{β}; and —OCOR^{β}.

In one embodiment, R¹, R², and R⁵, independently, are selected from —OH, and —OCH₃. For example, R³, R⁴, R⁶, R⁷, R⁸, and R⁹, are independently selected from H; halo; —CN; —NO₂; —R^{β}; —NH₂; —NHR^{β}; —N(RD)₂; —CHO; —COR^{β}; —COOH; —COOR^{β}; and —OCOR^{β}.

In one embodiment, R¹, R², and R⁵, independently, are selected from —OH, and —OCH₃. For example, R³, R⁴, R⁶, R⁷, R⁸, and R⁹, are independently selected from H; halo; —CN; —NO₂; and —NH₂.

In one embodiment, R¹, R², and R⁵, independently, are selected from —OH, and —OCH₃. For example, R³, R⁴, R⁶, R⁷, R⁸, and R⁹, are H.

In one embodiment, R¹, R² and R⁵, independently, are selected from —OH, —O—C₁₋₄ alkyl, —OC(O)R₁₃, —OC(O)NHR¹³, —OC(O)N(R¹³)₂; and R³, R⁴, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. For example, R³, R⁴, R⁶, R⁷, R⁸, and R⁹, independently, are selected from H;
halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; and benzyl optionally substituted with 1-3 —R. For example, R³, R⁴, R⁶, R⁷, R⁸, and R⁹, independently, are selected from H; halo; —CN; —NO₂; —SH; —SO₂H; —NH₂; —CHO; —COOH. For example, R³, R⁴, R⁶, R⁷, R⁸, and R⁹ are H.

In one embodiment, R¹, R² and R⁵ are independently selected from —OH and —O—C₁₋₄ alkyl, e.g. —OH and —OCH₃; and R³, R⁴, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. For example, R³, R⁴, R⁶, R⁷, R⁸, and R⁹, independently, are selected from H;
halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; and benzyl optionally substituted with 1-3 —R^{β}. For example, R³, R⁴, R⁶, R⁷, R⁸, and R⁹, independently, are selected from H; halo; —CN; —NO₂; —SH; —SO₂H; —NH₂; —CHO; —COOH. For example, R³, R⁴, R⁶, R⁷, R⁸, and R⁹ are H.

In one embodiment, R¹, R² and R⁵, independently, are selected from —OH and —O—C₁₋₄ alkyl; and R³, R⁴, R⁶, R⁷, R⁸, and R⁹, independently, are selected from H; halo; —CN; —NO₂; —SH; —SO₂H; and —NH₂. For example, R³, R⁴, R⁶, R⁷, R⁸, and R⁹ are H.

In one embodiment, R¹, R² and R⁵, independently, are selected from —OH and —OCH₃; and R³, R⁴, R⁶, R⁷, R⁸, and R⁹, independently, are selected from H;
halo; —CN; —NO₂; —SH; —SO₂H; and —NH₂. For example, R³, R⁴, R⁶, R⁷, R⁸, and R⁹ are H.

In one embodiment, R¹ is —O—C₁₋₄ alkyl, e.g. —O-Me; R² is —OH; R⁵ is —OH; and R³, R⁴, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. For example, R³, R⁴, R⁶, R⁷, R⁸, and R⁹, independently, are selected from H;
halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; and benzyl optionally substituted with 1-3 —R^{β}. For example, R³, R⁴, R⁶, R⁷, R⁸, and R⁹, independently, are selected from H; halo; —CN; —NO₂; —SH; —SO₂H; —NH₂; —CHO; —COOH. For example, R³, R⁴, R⁶, R⁷, R⁸, and R⁹ are H.

In one embodiment, R¹, R², and R⁵ are —OH; and R³, R⁴, R⁶, R⁷, R⁸, and R⁹, independently, are selected from H; halo; —CN; —NO₂; —R^{β}; —OH, —OR^{β}; —SH; —SR; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. For example, R³, R⁴, R⁶, R⁷, R⁸, and R⁹, independently, are selected from H;
halo; —CN; —NO₂; —R^{β}; —SH; —SR^{β}; —SOR^{β}; —SO₂H; —SO₂R; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; and benzyl optionally substituted with 1-3 —R^{β}. For example, R³, R⁴, R⁶, R⁷, R⁸, and R⁹, independently, are selected from H; halo; —CN; —NO₂; —SH; —SO₂H; —NH₂; —CHO; —COOH. For example, R³, R⁴, R⁶, R⁷, R⁸, and R⁹ are H.

In one embodiment, Z is —NR¹¹R¹².

In one embodiment, Z is —N(R¹⁰)—(CH₂)ₚ—NR¹¹R¹².

In one embodiment, Z is —N(R¹⁰)—(CH₂)_{q}—N(R¹⁰)—(CH₂)_{q}—NR¹¹R¹².

In one embodiment, Z is —N(R¹⁰)—(CH₂)_{q}—N(R¹⁰)—(CH₂)_{q}—N(R¹⁰)—(CH₂)_{q}—NR¹¹R¹².

Each R¹⁰ is independently selected from H, C₁₋₆ alkyl, C₂-C₆ alkenyl, C₂₋₆ alkynyl, C₃₋₁₀ cycloalkyl, and benzyl, wherein each R¹⁰, when not H, is independently optionally substituted with 1 or 2 —R^{β}.

For example, each R¹⁰ may independently be selected from H, C₁₋₆ alkyl, and C₂-C₄ alkenyl.

For example, each R¹⁰ may independently be selected from H, C₁₋₃ alkyl, and C₂-C₄ alkenyl.

For example, each R¹⁰ is independently selected from H and C₁₋₆ alkyl.

For example, each R¹⁰ may independently be selected from H and C₁₋₃ alkyl.

For example R¹⁰ may independently be selected from H and —CH₃.

R¹¹ and R¹² are independently selected from H, C₁₋₆-alkyl (e.g. methyl or ethyl), C₂-C₆ alkenyl, C₂₋₆ alkynyl, C₃₋₁₀ cycloalkyl (e.g. adamantyl), and benzyl, wherein each R¹¹ and R¹², when not H, are independently optionally substituted with 1 or 2 —R^{β}; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl (e.g. methyl) or benzyl.

In one embodiment, R¹¹ and R¹² are independently selected from H and C₁₋₆ alkyl (e.g. methyl or ethyl), C₃₋₁₀ cycloalkyl (e.g. adamantyl), and benzyl; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl (e.g. methyl) or benzyl.

In one embodiment, R¹¹ and R¹² are independently selected from H and C₁₋₆ alkyl; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl.

For example, R¹¹ and R¹² are independently selected from H and C₁₋₆ alkyl (e.g. methyl or ethyl), C₃₋₁₀ cycloalkyl (e.g. adamantyl), and benzyl; wherein each R¹¹ and R¹², when not H, are independently optionally substituted with 1 or 2 —R^{β}.

For example, R¹¹ is H, and R¹² is selected from H and C₁₋₆ alkyl (methyl or ethyl), C₃₋₁₀ cycloalkyl (e.g. adamantyl), and benzyl; wherein R¹², when not H, is optionally substituted with 1 —R^{β}.

For example, R¹¹ and R¹² are both H.

For example, R¹¹ is H, and R¹² is benzyl, optionally substituted with 1 —R^{β}. For example, R¹¹ is H, and R¹² is benzyl substituted with methoxy, e.g. R¹² is ortho-methoxy-benzyl. When R¹¹ and R¹² together form a 5- or 6-membered heterocycle as described above, it may be a 5- or 6-membered heterocycle optionally having one additional heteroatom selected from N and O; wherein the 5- or 6-membered heterocycle is optionally substituted with 1 or 2 C₁₋₄ alkyl. In this respect, the 5- or 6-membered heterocycle may be morpholine, piperidine, piperazine, or pyrrolidine optionally substituted with 1 or 2 C₁₋₄ alkyl. For example, the 5- or 6-membered heterocycle may be piperazine, 4-methyl piperazine, or pyrrolidine.

Each —R^{β} is independently selected from a C₁-C₆ alkyl, C₂-C₆ alkenyl, C₂-C₆ alkynyl, —O(C₁₋₂ alkyl) or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups.

For example, each —R^{β} is independently selected from a C₁-C₃ alkyl and —O(C₁-C₂ alkyl), and any —R^{β} may optionally be substituted with one or more halo, —OH, —NH₂, —CN, —NO₂, —C≡CH, —CHO, —CON(CH₃)₂ or oxo (═O) groups.

For example, each —R^{β} is independently selected from a C₁-C₃ alkyl and —O(C₁-C₂ alkyl), and any —R^{β} may optionally be substituted with one or more halo, —OH, —NH₂, —CN, —NO₂, —C≡CH, —CHO, —CON(CH₃)₂ or oxo (═O) groups.

For example, each —R^{β} is independently selected from C₁-C₂ alkyl and —O(C₁-C₂ alkyl), and any —R^{β} may optionally be substituted with one or more halo, —OH, —NH₂, —CN, or —NO₂ groups.

Each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴.

For example, each R¹³ is independently selected from a H and C₁-C₃ alkyl, wherein any —R¹³ may optionally be substituted with one or more —R¹⁴.

For example, each R¹³ is independently selected from a H and C₁-C₂ alkyl.

Each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅.

For example, each R¹⁴ is independently selected from a C₁-C₆ alkyl, halo, —NO₂, —CN, —OH, —NH₂, mercapto, formyl, carboxy, carbamoyl, and C₁₋₆ alkoxy, wherein any —R₁₄ may optionally be substituted with one or more —R₁₅.

Each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl.

For example, each —R¹⁵ is independently selected from halogen, nitro, cyano, hydroxy, trifluoromethoxy, trifluoromethyl, amino, formyl, and carboxy.

Each p is independently an integer selected from 1 to 4.

For example, each p is independently an integer selected from 2 to 4.

For example, each p is independently selected from 3 and 4.

Each q is independently an integer selected from 1 to 4.

For example, each q is independently an integer selected from 2 to 4.

For example, each q is independently selected from 3 and 4.

For example, Z may be —N(R¹⁰)—(CH₂)₃—N(R¹⁰)—(CH₂)₄—NR¹¹R¹².

For example, Z may be —N(R¹⁰)—(CH₂)₄—N(R¹⁰)—(CH₂)₃—NR¹¹R¹².

Each r is independently an integer selected from 1 to 4.

For example, each r is independently an integer selected from 2 to 4.

For example, each r is independently selected from 3 and 4.

For example, Z may be —N(R¹⁰)—(CH₂)ᵣ—N(R¹⁰)—(CH₂)ᵣ—N(R¹⁰)—(CH₂)ᵣ—NR¹¹R¹².

In one embodiment, Z is —NR¹¹R¹². For example, Z is —NR¹¹R¹²; R¹¹ and R¹² are independently selected from H; C₁₋₆ alkyl; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl.

In one embodiment, Z is —N(R¹⁰)—(CH₂)ₚ—NR¹¹R¹². For example, Z is —N(R¹⁰)—(CH₂)ₚ—NR¹¹R¹²; R¹⁰ is H or C₁₋₆ alkyl; and R¹¹ and R¹² are independently selected from H; C₁₋₆ alkyl; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl.

In one embodiment, Z is —N(R¹⁰)—(CH₂)ₚ—NR¹¹R¹²; and p is 1-4. For example, p is 2-4, for example 2 or 3.

In one embodiment, Z is —N(R¹⁰)—(CH₂)_{q}—N(R¹⁰)—(CH₂)_{q}—NR¹¹R¹²; and q is independently selected from 1-4. For example, q may be 2, 3 or 4. For example, Z is —N(R¹⁰)—(CH₂)_{q}—N(R¹⁰)—(CH₂)_{q}—NR¹¹R¹²; each R¹⁰ is independently selected from H and C₁₋₆ alkyl; and and R¹¹ and R¹² are independently selected from H; C₁₋₆ alkyl; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl.

In one embodiment, R³, R⁴, R⁷, R⁸ and R⁹ are H, for example as represented by Formula 1A below.

In one embodiment, the compound may be a compound of Formula 1A:

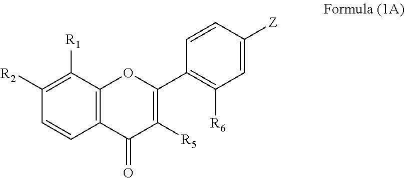

wherein R¹, R², R⁵, R⁶ and Z are as defined herein.

For example, the compound may be a compound of Formula (1A) wherein:
Z is selected from: —NR¹¹R¹²;
   —N(R¹⁰)—(CH₂)ₚ—NR¹¹R¹²;
   —N(R¹⁰)—(CH₂)_{q}—N(R¹⁰)—(CH₂)_{q}—NR¹¹R¹²; and
   —N(R¹⁰)—(CH₂)ᵣ—N(R¹⁰)—(CH₂)ᵣ—N(R¹⁰)—(CH₂)ᵣ—NR¹¹R¹²;
R¹, R², and R⁵ independently, are selected from —OH, —O—C₁₋₄ alkyl, —OC(O)R₁₃, —OC(O)NHR¹³, —OC(O)N(R¹³)₂; or R¹ and R² together form —O—CH₂—O—;
R³, R⁴, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β};
each —R^{β} is independently selected from a C₁-C₆ alkyl, C₂-C₆ alkenyl, C₂-C₆ alkynyl, —O(C₁₋₂ alkyl) or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
each R¹⁰ is independently selected from H, C₁₋₆ alkyl, C₂-C₆ alkenyl, C₂₋₆ alkynyl, C₃₋₁₀ cycloalkyl, and benzyl, wherein each R¹⁰, when not H, is independently optionally substituted with 1 or 2 —R^{β}; R¹¹ and R¹² are independently selected from H, C₁₋₆-alkyl, C₂-C₆ alkenyl, C₂₋₆ alkynyl, C₃₋₁₀ cycloalkyl, and benzyl, wherein each R¹¹ and R¹², when is not H, are independently optionally substituted with 1 or 2 —R^{β}; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl or benzyl;
each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl;
each p is independently an integer selected from 1 to 4; and each q is independently an integer selected from 1 to 4.

R¹, R², and R⁵, independently, are selected from —OH, —O—C₁₋₄ alkyl, —OC(O)R₁₃, —OC(O)NHR¹³, —OC(O)N(R¹³)₂. In one embodiment, R¹, R², and R⁵, independently, are selected from —OH, and —O—C₁₋₄ alkyl. In one embodiment, R¹, R², and R⁵, independently, are selected from —OH and —OCH₃.

In one embodiment, R¹ and R² together form —O—CH₂—O—.

R⁶ is selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. In one embodiment, R⁶ is selected from H;
halo; —CN; —NO₂; —R; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOR^{β}; and benzyl optionally substituted with 1-3 —R^{β}. In one embodiment, R⁶ is selected from H;
halo; —CN; —NO₂; —R; —OH; —OR^{β}; —NH₂; —NHR^{β}; —N(R^{β})₂; —CHO; —COR^{β}; —COOH; —COOR^{β}; and —OCOR^{β}. In one embodiment, R⁶ is selected from H;
halo; —CN; —NO₂; —R^{β}; —NH₂; —NHR^{β}; —N(R^{β})₂; —CHO; —COR^{β}; —COOH; —COOR^{β}; and —OCOR^{β}. In one embodiment, R⁶ is selected from H;
halo; —CN; —NO₂; —R^{β}; —NH₂; —NHR^{β}; —N(R^{β})₂; —CHO; —COR^{β}; —COOH; and —COOR^{β}. In one embodiment, R⁶ is selected from H; halo; —CN; —NO₂; and —NH₂. In one embodiment, R⁶ is H.

In one embodiment, R¹, R², and R⁵, independently, are selected from —OH, —O—C₁₋₄ alkyl, —OC(O)R₁₃, —OC(O)NHR¹³, —OC(O)N(R¹³)₂; and R⁶ is selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}.

In one embodiment, R¹, R², and R⁵, independently, are selected from —OH, and —O—C₁₋₄ alkyl. For example, R⁶ is selected from H;
halo; —CN; —NO₂; —R^{β}; —OH; —OR^{β}; —NH₂; —NHR^{β}; —N(RD)₂; —CHO; —COR^{β}; —COOH; —COOR^{β}; and —OCOR^{β}.

In one embodiment, R¹, R², and R⁵, independently, are selected from —OH, and —O—C₁₋₄ alkyl. For example, R⁶ is selected from H;
halo; —CN; —NO₂; —R^{β}; —NH₂; —NHR^{β}; —N(RD)₂; —CHO; —COR^{β}; —COOH; —COOR^{β}; and —OCOR^{β}.

In one embodiment, R¹, R², and R⁵, independently, are selected from —OH, and —OCH₃.

For example, R⁶ is selected from H;
halo; —CN; —NO₂; —R^{β}; —NH₂; —NHR^{β}; —N(RD)₂; —CHO; —COR^{β}; —COOH; —COOR^{β}; and —OCOR^{β}.

In one embodiment, R¹, R², and R⁵, independently, are selected from —OH, and —OCH₃.

For example, R⁶ is selected from H; halo; —CN; —NO₂; and —NH₂.

In one embodiment, R¹, R², and R⁵, independently, are selected from —OH, and —OCH₃. For example, R⁶ is H.

In one embodiment, R¹, R² and R⁵, independently, are selected from —OH, —O—C₁₋₄ alkyl, —OC(O)R₁₃, —OC(O)NHR¹³, —OC(O)N(R¹³)₂; and R⁶ is selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. For example, R⁶ is selected from H;
halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; and benzyl optionally substituted with 1-3 —R^{β}. For example, R⁶ is selected from H;
halo; —CN; —NO₂; —SH; —SO₂H; —NH₂; —CHO; —COOH. For example, R⁶ is H.

In one embodiment, R¹, R² and R⁵ are independently selected from —OH and —O—C₁₋₄ alkyl, e.g. —OH and —OCH₃; and R⁶ is selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. For example, R⁶ is selected from H;
halo; —CN; —NO₂; —R^{β}; —SH; —SR; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; and benzyl optionally substituted with 1-3 —R^{β}. For example, R⁶ is selected from H;
halo; —CN; —NO₂; —SH; —SO₂H; —NH₂; —CHO; —COOH. For example, R⁶ is H.

In one embodiment, R¹, R² and R⁵, independently, are selected from —OH and —O—C₁₋₄ alkyl; and R⁶ is selected from H; halo; —CN; —NO₂; —SH; —SO₂H; and —NH₂. For example, R⁶ is H.

In one embodiment, R¹, R² and R⁵, independently, are selected from —OH and —OCH₃; and R⁶ is selected from H; halo; —CN; —NO₂; —SH; —SO₂H; and —NH₂. For example, R⁶ is H.

In one embodiment, R¹ is —O—C₁₋₄ alkyl, e.g. —O-Me; R² is —OH; R⁵ is —OH; and R⁶ is selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. For example, R⁶ is selected from H;
halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; and benzyl optionally substituted with 1-3 —R^{β}. For example, R⁶ is selected from H;
halo; —CN; —NO₂; —SH; —SO₂H; —NH₂; —CHO; —COOH. For example, R⁶ is H.

In one embodiment, R¹, R², and R⁵ are —OH; and R⁶ is selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. For example, R⁶ is selected from H;
halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; and benzyl optionally substituted with 1-3 —R^{β}. For example, R⁶ is selected from H;
halo; —CN; —NO₂; —SH; —SO₂H; —NH₂; —CHO; —COOH. For example, R⁶ is H.

In one embodiment, Z is —NR¹¹R¹².

In one embodiment, Z is —N(R¹⁰)—(CH₂)ₚ—NR¹¹R¹².

In one embodiment, Z is —N(R¹⁰)—(CH₂)_{q}—N(R¹⁰)—(CH₂)_{q}—NR¹¹R¹².

In one embodiment, Z is —N(R¹⁰)—(CH₂)_{q}—N(R¹⁰)—(CH₂)_{q}—N(R¹⁰)—(CH₂)_{q}—NR¹¹R¹².

Each R¹⁰ is independently selected from H, C₁₋₆ alkyl, C₂-C₆ alkenyl, C₂₋₆ alkynyl, C₃₋₁₀ cycloalkyl, and benzyl, wherein each R¹⁰, when not H, is independently optionally substituted with 1 or 2 —R^{β}.

For example, each R¹⁰ may independently be selected from H, C₁₋₆ alkyl, and C₂-C₄ alkenyl.

For example, each R¹⁰ may independently be selected from H, C₁₋₃ alkyl, and C₂-C₄ alkenyl.

For example, each R¹⁰ is independently selected from H and C₁₋₆ alkyl.

For example, each R¹⁰ may independently be selected from H and C₁₋₃ alkyl.

For example, each R¹⁰ may independently be selected from H and —CH₃.

R¹¹ and R¹² are independently selected from H, C₁₋₆-alkyl (e.g. methyl or ethyl), C₂-C₆ alkenyl, C₂₋₆ alkynyl, C₃₋₁₀ cycloalkyl (e.g. adamantyl), and benzyl, wherein each R¹¹ and R¹², when not H, are independently optionally substituted with 1 or 2 —R^{β}; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl (e.g. methyl) or benzyl.

In one embodiment, R¹¹ and R¹² are independently selected from H and C₁₋₆ alkyl (e.g. methyl or ethyl), C₃₋₁₀ cycloalkyl (e.g. adamantyl), and benzyl; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl (e.g. methyl) or benzyl.

In one embodiment, R¹¹ and R¹² are independently selected from H and C₁₋₆ alkyl; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl.

For example, R¹¹ and R¹² are independently selected from H and C₁₋₆ alkyl (e.g. methyl or ethyl), C₃₋₁₀ cycloalkyl (e.g. adamantyl), and benzyl; wherein each R¹¹ and R¹², when not H, are independently optionally substituted with 1 or 2 —R^{β}.

For example, R¹¹ is H, and R¹² is selected from H and C₁₋₆ alkyl (methyl or ethyl), C₃₋₁₀ cycloalkyl (e.g. adamantyl), and benzyl; wherein R¹², when not H, is optionally substituted with 1 —R^{β}.

For example, R¹¹ and R¹² are both H.

For example, R¹¹ is H, and R¹² is benzyl, optionally substituted with 1 —R^{β}. For example, R¹¹ is H, and R¹² is benzyl substituted with methoxy, e.g. R¹² is ortho-methoxy-benzyl. When R¹¹ and R¹² together form a 5- or 6-membered heterocycle as described above, it may be a 5- or 6-membered heterocycle optionally having one additional heteroatom selected from N and O; wherein the 5- or 6-membered heterocycle is optionally substituted with 1 or 2 C₁₋₄ alkyl. In this respect, the 5- or 6-membered heterocycle may be morpholine, piperidine, piperazine, or pyrrolidine optionally substituted with 1 or 2 C₁₋₄ alkyl. For example, the 5- or 6-membered heterocycle may be piperazine, 4-methyl piperazine, or pyrrolidine.

Each —R^{β} is independently selected from a C₁-C₆ alkyl, C₂-C₆ alkenyl, C₂-C₆ alkynyl, —O(C₁₋₂ alkyl) or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups.

For example, each —R^{β} is independently selected from a C₁-C₃ alkyl and —O(C₁-C₂ alkyl), and any —R^{β} may optionally be substituted with one or more halo, —OH, —NH₂, —CN, —NO₂, —C≡CH, —CHO, —CON(CH₃)₂ or oxo (═O) groups.

For example, each —R^{β} is independently selected from a C₁-C₃ alkyl and —O(C₁-C₂ alkyl), and any —R^{β} may optionally be substituted with one or more halo, —OH, —NH₂, —CN, —NO₂, —C≡CH, —CHO, —CON(CH₃)₂ or oxo (═O) groups.

For example, each —R^{β} is independently selected from C₁-C₂ alkyl and —O(C₁-C₂ alkyl), and any —R^{β} may optionally be substituted with one or more halo, —OH, —NH₂, —CN, or —NO₂ groups.

Each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴.

For example, each R¹³ is independently selected from a H and C₁-C₃ alkyl, wherein any —R¹³ may optionally be substituted with one or more —R¹⁴.

For example, each R¹³ is independently selected from a H and C₁-C₂ alkyl.

Each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅.

For example, each R¹⁴ is independently selected from a C₁-C₆ alkyl, halo, —NO₂, —CN, —OH, —NH₂, mercapto, formyl, carboxy, carbamoyl, and C₁₋₆ alkoxy, wherein any —R₁₄ may optionally be substituted with one or more —R₁₅.

Each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl.

For example, each —R¹⁵ is independently selected from halogen, nitro, cyano, hydroxy, trifluoromethoxy, trifluoromethyl, amino, formyl, and carboxy.

Each p is independently an integer selected from 1 to 4.

For example, each p is independently an integer selected from 2 to 4.

For example, each p is independently selected from 3 and 4.

Each q is independently an integer selected from 1 to 4.

For example, each q is independently an integer selected from 2 to 4.

For example, each q is independently selected from 3 and 4.

For example, Z may be —N(R¹⁰)—(CH₂)₃—N(R¹⁰)—(CH₂)₄—NR¹¹R¹².

For example, Z may be —N(R¹⁰)—(CH₂)₄—N(R¹⁰)—(CH₂)₃—NR¹¹R¹².

Each r is independently an integer selected from 1 to 4.

For example, each r is independently an integer selected from 2 to 4.

For example, each r is independently selected from 3 and 4.

For example, Z may be —N(R¹⁰)—(CH₂)ᵣ—N(R¹⁰)—(CH₂)ᵣ—N(R¹⁰)—(CH₂)ᵣ—NR¹¹R¹².

In one embodiment, Z is —NR¹¹R¹². For example, Z is —NR¹¹R¹²; R¹¹ and R¹² are independently selected from H; C₁₋₆ alkyl; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl.

In one embodiment, Z is —N(R¹⁰)—(CH₂)ₚNR¹¹R¹². For example, Z is —N(R¹⁰)—(CH₂)ₚ—NR¹¹R¹²; R¹⁰ is H or C₁₋₆ alkyl; and R¹¹ and R¹² are independently selected from H; C₁₋₆ alkyl; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl.

In one embodiment, Z is —N(R¹⁰)—(CH₂)ₚ—NR¹¹R¹²; and p is 1-4. For example, p is 2-4, for example 2 or 3.

In one embodiment, Z is —N(R¹⁰)—(CH₂)_{q}—N(R¹⁰)—(CH₂)_{q}—NRⁿR¹²; and q is independently selected from 1-4. For example, q may be 2, 3 or 4. For example, Z is —N(R¹⁰)—(CH₂)_{q}—N(R¹⁰)—(CH₂)_{q}—NR¹¹R¹²; each R¹⁰ is independently selected from H and C₁₋₆ alkyl; and R¹¹ and R¹² are independently selected from H; C₁₋₆ alkyl; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl.

A second aspect of the invention provides a compound selected from compounds listed in Table A.

TABLE A

| | |
|---|---|
| 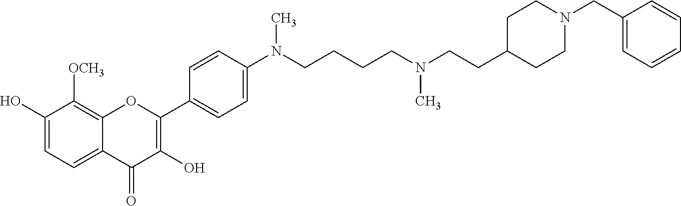 [SMILES: COc1c(O)ccc2c(=O)c(O)c(-c3ccc(N(C)CCCCN(C)CCC4CCN(Cc5ccccc5)CC4)cc3)oc12] | SND 437 |
| 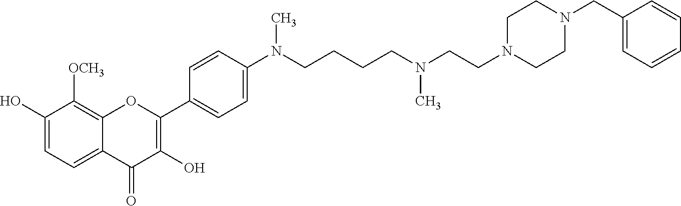 [SMILES: COc1c(O)ccc2c(=O)c(O)c(-c3ccc(N(C)CCCCN(C)CCN4CCN(Cc5ccccc5)CC4)cc3)oc12] | SND 438 |
| 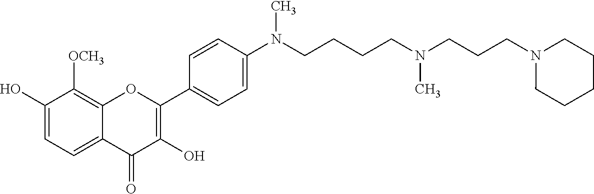 [SMILES: COc1c(O)ccc2c(=O)c(O)c(-c3ccc(N(C)CCCCN(C)CCCN4CCCCC4)cc3)oc12] | SND 439 |
| 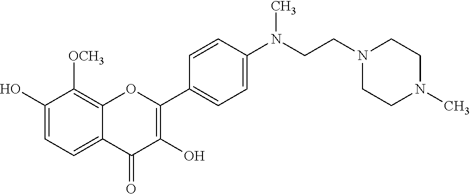 [SMILES: COc1c(O)ccc2c(=O)c(O)c(-c3ccc(N(C)CCN4CCN(C)CC4)cc3)oc12] | SND 440 |
| 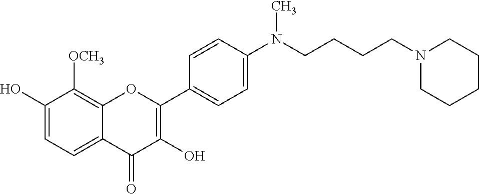 [SMILES: COc1c(O)ccc2c(=O)c(O)c(-c3ccc(N(C)CCCCN4CCCCC4)cc3)oc12] | SND 441 |
| 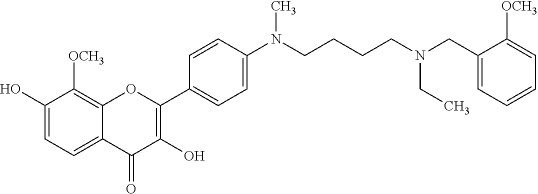 [SMILES: CCN(CCCCN(C)c1ccc(-c2oc3c(OC)c(O)ccc3c(=O)c2O)cc1)Cc1ccccc1OC] | SND 443 |
| 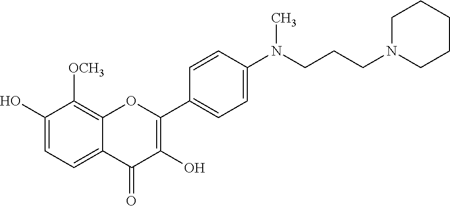 [SMILES: COc1c(O)ccc2c(=O)c(O)c(-c3ccc(N(C)CCCN4CCCCC4)cc3)oc12] | SND 444 |
| 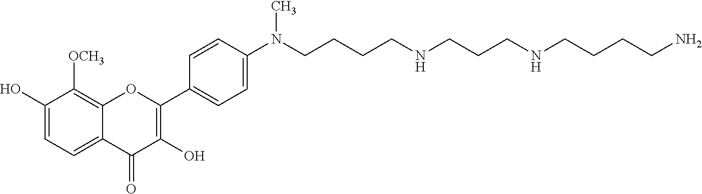 [SMILES: COc1c(O)ccc2c(=O)c(O)c(-c3ccc(N(C)CCCCNCCCNCCCCN)cc3)oc12] | SND 445 |
| 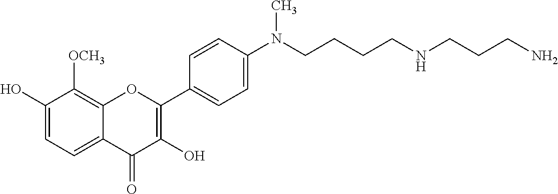 [SMILES: COc1c(O)ccc2c(=O)c(O)c(-c3ccc(N(C)CCCCNCCCN)cc3)oc12] | SND 446 |
| 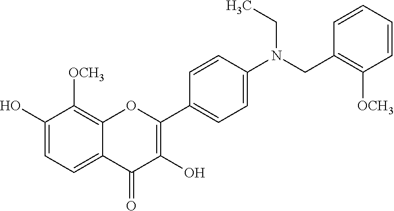 [SMILES: CCN(Cc1ccccc1OC)c1ccc(-c2oc3c(OC)c(O)ccc3c(=O)c2O)cc1] | SND 447 |
| 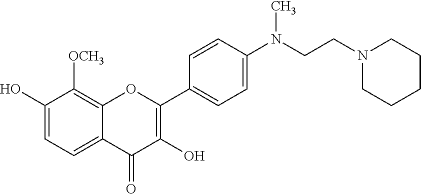 [SMILES: COc1c(O)ccc2c(=O)c(O)c(-c3ccc(N(C)CCN4CCCCC4)cc3)oc12] | SND 448 |
| 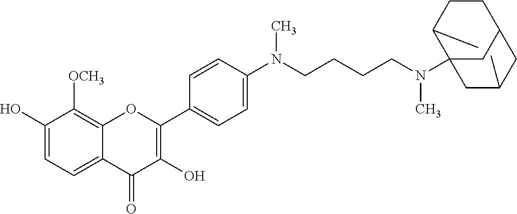 [SMILES: COc1c(O)ccc2c(=O)c(O)c(-c3ccc(N(C)CCCCN(C)C45CC(C)CC(CCC4C)C5)cc3)oc12] | SND 449 |
| 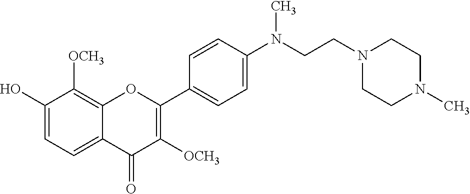 [SMILES: COc1c(-c2ccc(N(C)CCN3CCN(C)CC3)cc2)oc2c(OC)c(O)ccc2c1=O] | SND 450 |
| 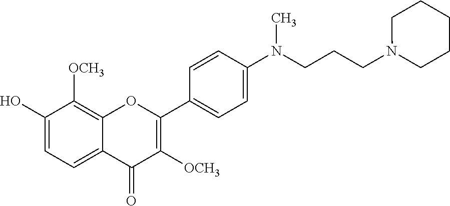 [SMILES: COc1c(-c2ccc(N(C)CCCN3CCCCC3)cc2)oc2c(OC)c(O)ccc2c1=O] | SND 451 |
| 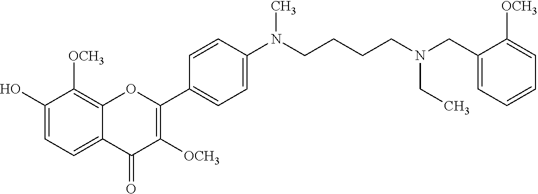 [SMILES: CCN(CCCCN(C)c1ccc(-c2oc3c(OC)c(O)ccc3c(=O)c2OC)cc1)Cc1ccccc1OC] | SND 453 |
| 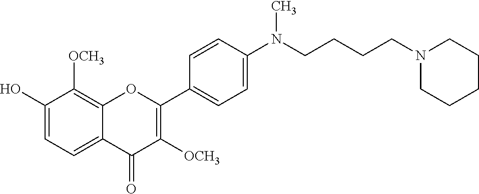 [SMILES: COc1c(-c2ccc(N(C)CCCCN3CCCCC3)cc2)oc2c(OC)c(O)ccc2c1=O] | SND 454 |
| 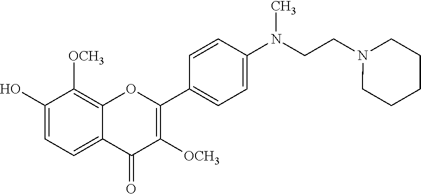 [SMILES: COc1c(-c2ccc(N(C)CCN3CCCCC3)cc2)oc2c(OC)c(O)ccc2c1=O] | SND 455 |
| 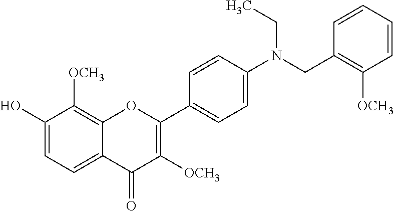 [SMILES: CCN(Cc1ccccc1OC)c1ccc(-c2oc3c(OC)c(O)ccc3c(=O)c2OC)cc1] | SND 456 |
| 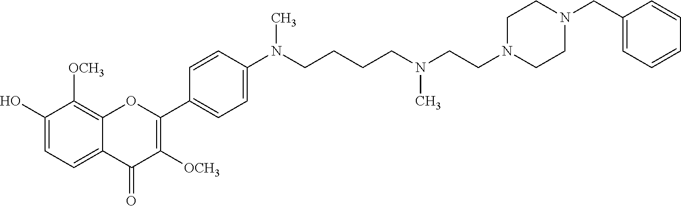 [SMILES: COc1c(-c2ccc(N(C)CCCCN(C)CCN3CCN(Cc4ccccc4)CC3)cc2)oc2c(OC)c(O)ccc2c1=O] | SND 457 |
| 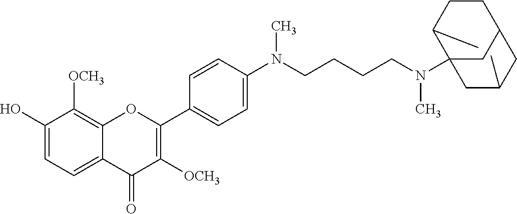 [SMILES: COc1c(-c2ccc(N(C)CCCCN(C)C34CC(C)CC(CCC3C)C4)cc2)oc2c(OC)c(O)ccc2c1=O] | SND 458 |
| 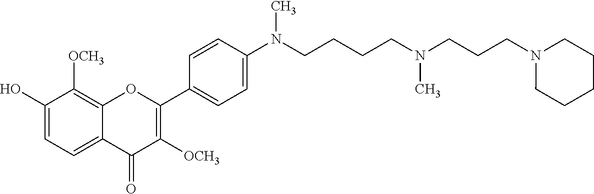 [SMILES: COc1c(-c2ccc(N(C)CCCCN(C)CCCN3CCCCC3)cc2)oc2c(OC)c(O)ccc2c1=O] | SND 459 |
| 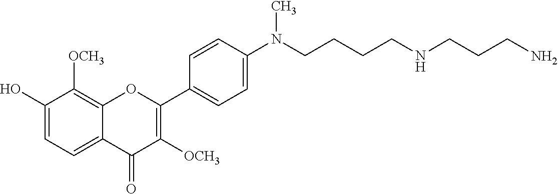 [SMILES: COc1c(-c2ccc(N(C)CCCCNCCCN)cc2)oc2c(OC)c(O)ccc2c1=O] | SND 461 |
| 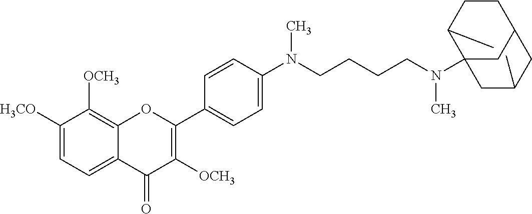 [SMILES: COc1ccc2c(=O)c(OC)c(-c3ccc(N(C)CCCCN(C)C45CC(C)CC(CCC4C)C5)cc3)oc2c1OC] | SND 462 |
| 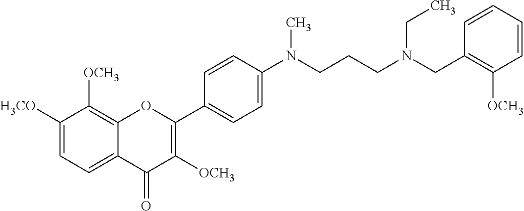 [SMILES: CCN(CCCN(C)c1ccc(-c2oc3c(OC)c(OC)ccc3c(=O)c2OC)cc1)Cc1ccccc1OC] | SND 463 |
| 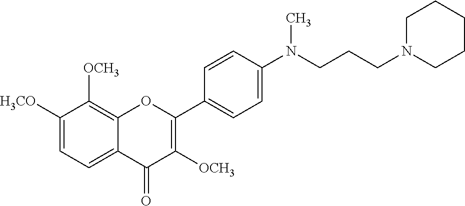 [SMILES: COc1ccc2c(=O)c(OC)c(-c3ccc(N(C)CCCN4CCCCC4)cc3)oc2c1OC] | SND 464 |
| 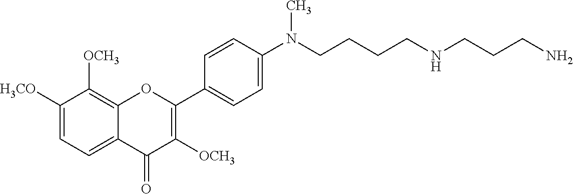 [SMILES: COc1ccc2c(=O)c(OC)c(-c3ccc(N(C)CCCCNCCCN)cc3)oc2c1OC] | SND 465 |

A third aspect of the invention provides pharmaceutically acceptable salt, multi-salt, solvate or prodrug of the compound of the first or second aspect of the invention.

The compounds of the present invention can be used both in their quaternary salt form (as a single salt). Additionally, the compounds of the present invention may contain one or more (e.g. one or two) acid addition or alkali addition salts to form a multi-salt. A multi-salt includes a quaternary salt group as well as a salt of a different group of the compound of the invention.

For the purposes of this invention, a “multi-salt” of a compound of the present invention includes an acid addition salt. Acid addition salts are preferably pharmaceutically acceptable, non-toxic addition salts with suitable acids, including but not limited to inorganic acids such as hydrohalogenic acids (for example, hydrofluoric, hydrochloric, hydrobromic or hydroiodic acid) or other inorganic acids (for example, nitric, perchloric, sulfuric or phosphoric acid); or organic acids such as organic carboxylic acids (for example, propionic, butyric, glycolic, lactic, mandelic, citric, acetic, benzoic, salicylic, succinic, malic or hydroxysuccinic, tartaric, fumaric, maleic, hydroxymaleic, mucic or galactaric, gluconic, pantothenic or pamoic acid), organic sulfonic acids (for example, methanesulfonic, trifluoromethanesulfonic, ethanesulfonic, 2-hydroxyethanesulfonic, benzenesulfonic, toluene-p-sulfonic, naphthalene-2-sulfonic or camphorsulfonic acid) or amino acids (for example, ornithinic, glutamic or aspartic acid). The acid addition salt may be a mono-, di-, tri- or multi-acid addition salt. A preferred salt is a hydrohalogenic, sulfuric, phosphoric or organic acid addition salt. A preferred salt is a hydrochloric acid addition salt.

The compounds of the present invention can be used both, in quaternary salt form and their multi-salt form. For the purposes of this invention, a “multi-salt” of a compound of the present invention includes one formed between a protic acid functionality (such as a carboxylic acid group) of a compound of the present invention and a suitable cation. Suitable cations include, but are not limited to lithium, sodium, potassium, magnesium, calcium and ammonium. The salt may be a mono-, di-, tri- or multi-salt. Preferably the salt is a mono- or di-lithium, sodium, potassium, magnesium, calcium or ammonium salt. More preferably the salt is a mono- or di-sodium salt or a mono- or di-potassium salt.

Preferably any multi-salt is a pharmaceutically acceptable non-toxic salt. However, in addition to pharmaceutically acceptable multi-salts, other salts are included in the present invention, since they have potential to serve as intermediates in the purification or preparation of other, for example, pharmaceutically acceptable salts, or are useful for identification, characterisation or purification of the free acid or base.

The compounds and/or multi-salts of the present invention may be anhydrous or in the form of a hydrate (e.g. a hemihydrate, monohydrate, dihydrate or trihydrate) or other solvate. Such solvates may be formed with common organic solvents, including but not limited to, alcoholic solvents e.g. methanol, ethanol or isopropanol.

In some embodiments of the present invention, therapeutically inactive prodrugs are provided. Prodrugs are compounds which, when administered to a subject such as a human, are converted in whole or in part to a compound of the invention. In most embodiments, the prodrugs are pharmacologically inert chemical derivatives that can be converted in vivo to the active drug molecules to exert a therapeutic effect. Any of the compounds described herein can be administered as a prodrug to increase the activity, bioavailability, or stability of the compound or to otherwise alter the properties of the compound. Typical examples of prodrugs include compounds that have biologically labile protecting groups on a functional moiety of the active compound. Prodrugs include, but are not limited to, compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound. The present invention also encompasses multi-salts and solvates of such prodrugs as described above.

The compounds, multi-salts, solvates and prodrugs of the present invention may contain at least one chiral centre. The compounds, multi-salts, solvates and prodrugs may therefore exist in at least two isomeric forms. The present invention encompasses racemic mixtures of the compounds, multi-salts, solvates and prodrugs of the present invention as well as enantiomerically enriched and substantially enantiomerically pure isomers. For the purposes of this invention, a “substantially enantiomerically pure” isomer of a compound comprises less than 5% of other isomers of the same compound, more typically less than 2%, and most typically less than 0.5% by weight.

The compounds, multi-salts, solvates and prodrugs of the present invention may contain any stable isotope including, but not limited to ¹²C, ¹³C, ¹H, ²H (D), ¹⁴N, ¹⁵N, ¹⁶O, ¹⁷O, ¹⁸O, ¹⁹F and ¹²⁷I, and any radioisotope including, but not limited to ¹¹C, ¹⁴C, ³H (T), ¹³N, ¹⁵O, ¹⁸F, ¹²³I, ¹²⁴I, ¹²⁵I and ¹³¹I.

The compounds, multi-salts, solvates and prodrugs of the present invention may be in any polymorphic or amorphous form.

A fourth aspect of the invention provides a pharmaceutical composition comprising a compound of the first or second aspect of the invention, or a pharmaceutically acceptable multi-salt, solvate or prodrug of the third aspect of the invention, and a pharmaceutically acceptable excipient.

Conventional procedures for the selection and preparation of suitable pharmaceutical formulations are described in, for example, “Aulton's Pharmaceutics—The Design and Manufacture of Medicines”, M. E. Aulton and K. M. G. Taylor, Churchill Livingstone Elsevier, 4^{th} Ed., 2013.

Pharmaceutically acceptable excipients including adjuvants, diluents or carriers that may be used in the pharmaceutical compositions of the invention are those conventionally employed in the field of pharmaceutical formulation, and include, but are not limited to, sugars, sugar alcohols, starches, ion exchangers, alumina, aluminium stearate, lecithin, serum proteins such as human serum albumin, buffer substances such as phosphates, glycerine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinylpyrrolidone, cellulose-based substances, polyethylene glycol, sodium carboxymethylcellulose, polyacrylates, waxes, polyethylene-polyoxypropylene-block polymers, polyethylene glycol and wool fat.

A fifth aspect of the invention provides a compound of the first or second aspect of the invention, or a pharmaceutically acceptable multi-salt, solvate or prodrug of the third aspect of the invention, or a pharmaceutical composition of the fourth aspect of the invention, for use in medicine, and/or for use in the treatment or prevention of a disease, disorder or condition. Typically the use comprises the administration of the compound, multi-salt, solvate, prodrug or pharmaceutical composition to a subject. In one embodiment, the disease, disorder or condition is cancer.

A sixth aspect of the invention provides the use of a compound of the first or second aspect, a pharmaceutically effective multi-salt, solvate or prodrug of the third aspect, or a pharmaceutical composition according to the fourth aspect, in the manufacture of a medicament for the treatment or prevention of a disease, disorder or condition. Typically the treatment or prevention comprises the administration of the compound, multi-salt, solvate, prodrug or pharmaceutical composition to a subject. In one embodiment, the disease, disorder or condition is cancer.

A seventh aspect of the invention provides a method of treatment or prevention of a disease, disorder or condition, the method comprising the step of administering an effective amount of a compound of the first or second aspect, or a pharmaceutically acceptable multi-salt, solvate or prodrug of the third aspect, or a pharmaceutical composition of the fourth aspect, to thereby treat or prevent the disease, disorder or condition. Typically the administration is to a subject in need thereof. In one embodiment, the disease, disorder or condition is cancer.

The term “treatment” as used herein refers equally to curative therapy, and ameliorating or palliative therapy. The term includes obtaining beneficial or desired physiological results, which may or may not be established clinically. Beneficial or desired clinical results include, but are not limited to, the alleviation of symptoms, the prevention of symptoms, the diminishment of extent of disease, the stabilisation (i.e., not worsening) of a condition, the delay or slowing of progression/worsening of a condition/symptoms, the amelioration or palliation of the condition/symptoms, and remission (whether partial or total), whether detectable or undetectable. The term “palliation”, and variations thereof, as used herein, means that the extent and/or undesirable manifestations of a physiological condition or symptom are lessened and/or time course of the progression is slowed or lengthened, as compared to not administering a compound, multi-salt, solvate, prodrug or pharmaceutical composition of the present invention. The term “prevention” as used herein in relation to a disease, disorder or condition, relates to prophylactic or preventative therapy, as well as therapy to reduce the risk of developing the disease, disorder or condition. The term “prevention” includes both the avoidance of occurrence of the disease, disorder or condition, and the delay in onset of the disease, disorder or condition. Any statistically significant avoidance of occurrence, delay in onset or reduction in risk as measured by a controlled clinical trial may be deemed a prevention of the disease, disorder or condition. Subjects amenable to prevention include those at heightened risk of a disease, disorder or condition as identified by genetic or biochemical markers. Typically, the genetic or biochemical markers are appropriate to the disease, disorder or condition under consideration and may include for example, beta-amyloid 42, tau and phosphor-tau.

In one embodiment of the fifth, sixth, or seventh aspect of the present invention, the disease, disorder or condition is selected from but not limited to: breast cancer, brain cancer, colon cancer, lung cancer, leukaemia, lymphoma, muscle carcinoma, melanoma, renal cancer, prostate cancer and pancreatic cancer. The cancers may include resistant types of such tumors.

For example, the disease, disorder or condition is selected from but not limited to: brain cancer, colon cancer, leukaemia, lung cancer, lymphoma, muscle carcinoma, and melanoma.

In general embodiments, the disease, disorder or condition is cancer.

In one embodiment the cancer is brain cancer.

In one embodiment the cancer is breast cancer.

In one embodiment the cancer is colon cancer.

In one embodiment the cancer is leukaemia.

In one embodiment the cancer is lung cancer.

In one embodiment the cancer is lymphoma.

In one embodiment the cancer is muscle carcinoma.

In one embodiment the cancer is melanoma.

In one embodiment the cancer is ovarian cancer.

In one embodiment the cancer is pancreatic cancer.

In one embodiment the cancer is prostate cancer.

In one embodiment the cancer is renal cancer.

Unless stated otherwise, in any aspect of the invention, the subject may be any human or other animal. Typically, the subject is a mammal, more typically a human or a domesticated mammal such as a cow, pig, lamb, goat, horse, cat, dog, etc. Most typically, the subject is a human.

Any of the medicaments employed in the present invention can be administered by oral, parental (including intravenous, subcutaneous, intramuscular, intradermal, intratracheal, intraperitoneal, intraarticular, intracranial and epidural), airway (aerosol), rectal, vaginal or topical (including transdermal, buccal, mucosal and sublingual) administration.

Typically, the mode of administration selected is that most appropriate to the disorder or disease to be treated or prevented.

For oral administration, the compounds, multi-salts, solvates or prodrugs of the present invention will generally be provided in the form of tablets, capsules, hard or soft gelatine capsules, caplets, troches or lozenges, as a powder or granules, or as an aqueous solution, suspension or dispersion.

Tablets for oral use may include the active ingredient mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose. Corn starch and alginic acid are suitable disintegrating agents. Binding agents may include starch and gelatine. The lubricating agent, if present, may be magnesium stearate, stearic acid or tale. If desired, the tablets may be coated with a material, such as glyceryl monostearate or glyceryl distearate, to delay absorption in the gastrointestinal tract. Tablets may also be effervescent and/or dissolving tablets.

Capsules for oral use include hard gelatine capsules in which the active ingredient is mixed with a solid diluent, and soft gelatine capsules wherein the active ingredient is mixed with water or an oil such as peanut oil, liquid paraffin or olive oil.

Powders or granules for oral use may be provided in sachets or tubs. Aqueous solutions, suspensions or dispersions may be prepared by the addition of water to powders, granules or tablets.

Any form suitable for oral administration may optionally include sweetening agents such as sugar, flavouring agents, colouring agents and/or preservatives.

Formulations for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

For parenteral use, the compounds, multi-salts, solvates or prodrugs of the present invention will generally be provided in a sterile aqueous solution or suspension, buffered to an appropriate pH and isotonicity. Suitable aqueous vehicles include Ringer's solution and isotonic sodium chloride or glucose. Aqueous suspensions according to the invention may include suspending agents such as cellulose derivatives, sodium alginate, polyvinylpyrrolidone and gum tragacanth, and a wetting agent such as lecithin. Suitable preservatives for aqueous suspensions include ethyl and n-propyl p-hydroxybenzoate. The compounds of the invention may also be presented as liposome formulations.

For transdermal and other topical administration, the compounds, multi-salts, solvates or prodrugs of the invention will generally be provided in the form of ointments, cataplasms (poultices), pastes, powders, dressings, creams, plasters or patches.

Suitable suspensions and solutions can be used in inhalers for airway (aerosol) administration.

The dose of the compounds, multi-salts, solvates or prodrugs of the present invention will, of course, vary with the disorder or disease to be treated or prevented. In general, a suitable dose will be in the range of 0.01 to 500 mg per kilogram body weight of the recipient per day. The desired dose may be presented at an appropriate interval such as once every other day, once a day, twice a day, three times a day or four times a day. The desired dose may be administered in unit dosage form, for example, containing 1 mg to 50 g of active ingredient per unit dosage form.

For the avoidance of doubt, insofar as is practicable any embodiment of a given aspect of the present invention may occur in combination with any other embodiment of the same aspect of the present invention. In addition, insofar as is practicable it is to be understood that any preferred, typical or optional embodiment of any aspect of the present invention should also be considered as a preferred, typical or optional embodiment of any other aspect of the present invention.

### EXAMPLES

### Synthesis of Compound 006-004 (SND 448)

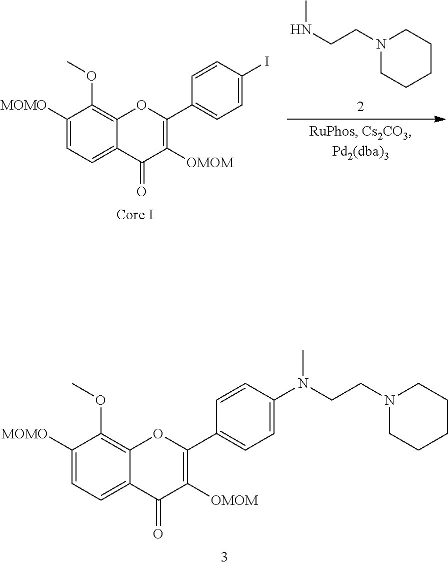

To a solution of Core I (300 mg, 602.09 umol, 1.00 eq), Compound 2 (85.64 mg, 602.09 umol, 1.00 eq) in DMF (3 mL) was added Cs₂CO₃ (588.52 mg, 1.81 mmol, 3.00 eq), RuPhos (28.10 mg, 60.21 umol, 0.10 eq), Pd₂(dba)₃ (55.13 mg, 60.21 umol, 0.10 eq), and the mixture was stirred at 100° C. for 16 h under N₂. LCMS showed Core I was consumed and desired mass was detected. The reaction mixture was filtered and concentrated under reduced pressure to give a residue. Compound 3(300 mg, crude) was obtained as a red oil.

LCMS: MS (ESI) Retention time: 0.714 min, (M+1)⁺=513.3.

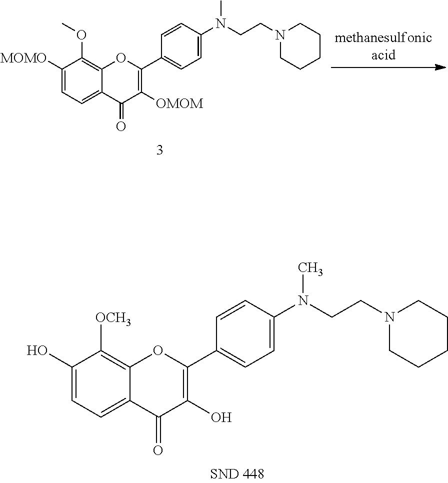

To a solution of Compound 3 (300 mg, 585.26 umol, 1.00 eq) in DCM (0.3 mL) and EtOH (2.1 mL) was added methanesulfonic acid (674.96 mg, 7.02 mmol, 499.97 uL, 12.0 eq), and the mixture was stirred at 60° C. or 1 h. LCMS showed Compound 3 was consumed and desired mass was detected. The reaction mixture was concentrated under reduced pressure to give a residue. The crude product was purified by reversed-phase HPLC (0.1% HCl condition). Compound 006-004 (SND 448) (180 mg, 382.69 umol, 65.39% yield, 98% purity, HCl) was obtained as a yellow solid, which was confirmed by LCMS and HNMR.

LCMS: MS (ESI) Retention time: 0.679 min, (M+1)⁺=425.2.

LCMS: MS (ESI) Retention time: 1.127 min, (M+1)⁺=425.1.

¹H NMR (400 MHz, DMSO-d6) δ=11.03 (s, 1H), 8.10-8.08 (d, J=9.2 Hz, 2H), 7.66-7.64 (d, J=8.8 Hz, 1H), 7.04-7.00 (t, J=9.2 Hz, 3H), 3.92 (s, 5H), 3.49-3.46 (d, J=11.2 Hz, 2H), 3.17-3.16 (d, J=2.8 Hz, 2H), 3.02 (s, 3H), 2.95-2.88 (m, 2H), 1.85-1.69 (m, 5H), 1.38-1.35 (m, 1H).

TLC (SiO2, DCM/MeOH=10/1, Rf=0.4).

### Synthesis of Compound 006-005 (SND 440)

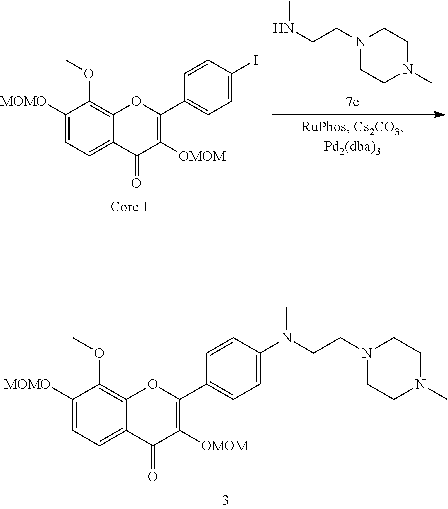

To a solution of Compound 7e (94.68 mg, 602.09 μmol, 5.72 μL, 1.00 eq) and Core I (300 mg, 602.09 μmol, 1.00 eq) in DMF (3 mL) was added RuPhos (56.19 mg, 120.42 μmol, 0.20 eq), Cs₂CO₃ (588.52 mg, 1.81 mmol, 3.00 eq) and Pd₂ (dba)₃ (110.27 mg, 120.42 μmol, 0.20 eq) at 25° C. The mixture was stirred at 100° C. for 12 hr under N₂. LCMS showed Core I was consumed. Several new peaks were shown on LCMS and 51.233% of desired compound was detected. The reaction mixture was filtered and concentrated under reduced pressure to give a residue. Without purification. It was put in the next reaction. Compound 3 (300 mg, 568.60 μmol, 94.44% yield) was obtained as a red oil.

LCMS: MS (ESI) Retention time: 0.691 min (M+H)⁺=528.4.

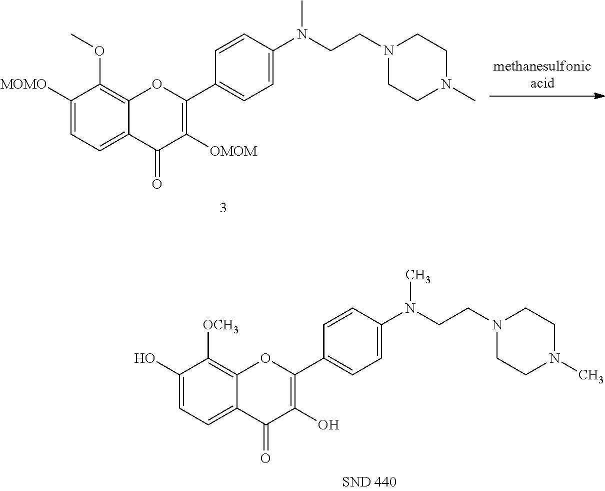

To a solution of Compound 3 (290 mg, 549.65 μmol, 1.00 eq) in EtOH (0.7 mL) and DCM (0.1 mL) was added methanesulfonic acid (633.92 mg, 6.60 mmol, 469.57 μL, 12 eq). The mixture was stirred at 60° C. for 1 hr. LCMS showed Compound 3 was consumed completely and 62.352% of desired mass was detected. The reaction mixture was concentrated under reduced pressure to give a residue. The crude product was purified by re-crystallization from MeOH (60 mL) at 25° C. Compound 006-005 (SND 440) (239.42 mg, 533.20 μmol, 97.01% yield, 97.88% purity) was obtained as a yellow solid, which was confirmed by HNMR, Melting Point and LCMS.

TLC (SiO₂, DCM: MeOH=10/1, Rf=0.1) and Melting Point=261.3° C.

LCMS: MS (ESI) Retention time: 0.656 min (M+H)⁺=440.2.

LCMS: MS (ESI) Retention time: 1.380 min (M+H)⁺=440.2.

¹H NMR (400 MHz, DMSO-d₆) δ=8.09-8.07 (d, J=9.2 Hz, 2H), 7.68-7.65 (d, J=8.8 Hz, 1H), 7.00-6.98 (d, J=8.8 Hz, 1H), 6.93-6.91 (d, J=9.2 Hz, 2H), 3.72-3.69 (t, J=7.0 Hz, 2H), 3.42-3.29 (m, 6H), 3.13-3.12 (t, J=6.5 Hz, 2H), 3.00 (s, 3H), 2.84 (s, 3H), 2.43 (s, 5H).

### Synthesis of Compound 006-006 (SND 447)

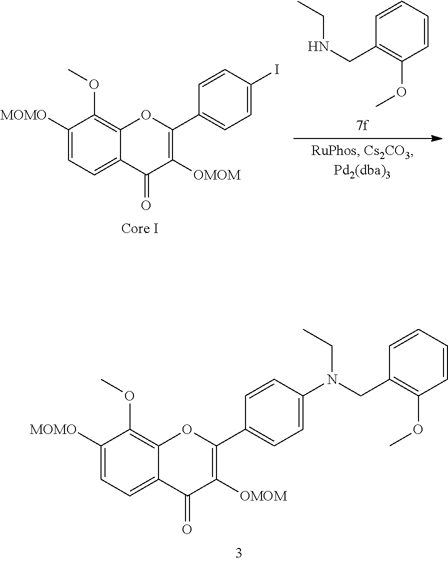

To a solution of Compound 7f (99.48 mg, 602.09 μmol, 5.72 μL, 1.00 eq) and Core I (300 mg, 602.09 μmol, 1000 eq) in DMF (3 mL) was added RuPhos (56.19 mg, 120.42 μmol, 0.20 eq), Cs₂CO₃ (588.52 mg, 1.81 mmol, 3.00 eq) and Pd₂ (dba)₃ (110.27 mg, 120.42 μmol, 0.20 eq) at 25° C. And that mixture was degassed and purged with N₂ for 3 times. The mixture was stirred at 100° C. for 12 hr under N₂ atmosphere. LCMS showed Core I was consumed. Several new peaks were shown on LCMS and 28.925% of desired compound was detected. The reaction mixture was concentrated under reduced pressure to give a residue. The residue was purified by prep-HPLC (column: Waters Xbridge C18 150×50 mmx 10 um; mobile phase: [water (NH₄HCO₃) -ACN]; B %: 500%-800%, 10 min). Compound 3 (100 mg, 186.71 μmol, 31.01% yield) was obtained as a red oil.

LCMS: MS (ESI) Retention time: 1.003 min (M+H)⁺=536.2.

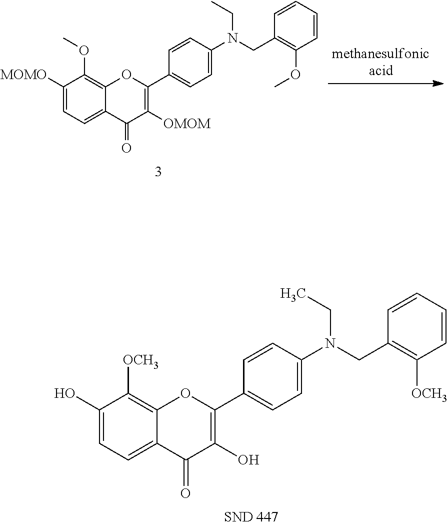

To a solution of Compound 3 (100 mg, 186.71 μmol, 1 eq) in MeOH (1.4 mL) and DCM (0.2 mL) was added methanesulfonic acid (215.33 mg, 2.24 mmol, 159.50 μL, 12 eq). The mixture was stirred at 60° C. for 1 hr. LCMS showed Compound 3 was consumed completely and 90.397% of desired mass was detected. The reaction mixture was concentrated under reduced pressure to give a residue. The residue was purified by prep-HPLC (column: 3_Phenomenex Luna C18; 75×30 mm×3 um; mobile phase: [water (HCl)-ACN]; B %: 51%-71%, 6 min). Compound 006-006 (SND 447) (90 mg, 185.97 μmol, 99.60% yield, 100% purity, HCl) was obtained as a yellow oil. Which was determined by HNMR and LCMS.

TLC (SiO₂, PE: EA=0/1, Rf=0.5).

LCMS: MS (ESI) Retention time: 0.960 min (M+H)⁺=448.2.

LCMS: MS (ESI) Retention time: 1.872 min (M+H)⁺=448.2.

¹H NMR (400 MHz, DMSO-d₆) δ=8.03-8.00 (d, J=9.2 Hz, 2H), 7.66-7.63 (d, J=8.8 Hz, 1H), 7.25 (m, 1H), 7.05-7.03 (d, J=8.4 Hz, 1H), 6.98-6.95 (m, 2H), 6.88-6.86 (m, 1H), 6.78-6.76 (d, J=8.4 Hz, 2H), 4.55 (s, 2H), 3.92 (s, 3H), 3.86 (s, 3H), 3.58-3.53 (q, J=6.8 Hz, 2H), 1.20-1.17 (t, J=7.2 Hz, 3H).

### Synthesis of Compound 006-007 (SND 441)

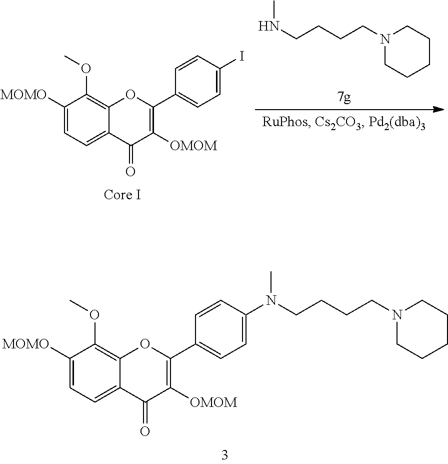

To a solution of Compound 7g (102.53 mg, 602.09 μmol, 5.72 μL, 1.00 eq) and Core I (300 mg, 602.09 μmol, 1.00 eq) in DMF (1 mL) was added RuPhos (56.19 mg, 120.42 μmol, 0.20 eq), Cs₂CO₃ (588.52 mg, 1.81 mmol, 3.00 eq) and Pd₂ (dba)₃ (110.27 mg, 120.42 μmol, 0.20 eq) at 25° C. The mixture was stirred at 100° C. for 12 hr. LCMS showed Core I was consumed. Several new peaks were shown on LCMS and 48.736% of desired compound was detected. The reaction mixture was filtered and concentrated under reduced pressure to give a residue. Without purification. It was put in the next reaction. Compound 3 (300 mg, 560.14 μmol, 93.03% yield) was obtained as a red oil.

LCMS: MS (ESI) Retention time: 0.740 min (M+H)⁺=541.4.

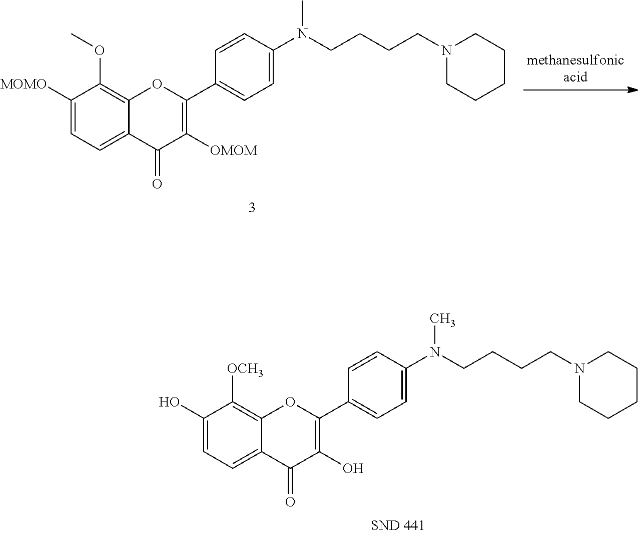

To a solution of Compound 3 (300 mg, 554.89 μmol, 1.00 eq) in EtOH (3.5 mL) and DCM (0.5 mL) was added methanesulfonic acid (639.94 mg, 6.66 mmol, 474.03 μL, 12 eq). The mixture was stirred at 60° C. for 0.5 hr. LCMS showed Compound 3 was consumed completely and 55.545% of desired mass was detected. The reaction mixture was concentrated under reduced pressure to give a residue. The crude product was purified by reversed-phase HPLC (0.1% HCl condition). Compound 006-007 (SND 441) (190 mg, 381.94 μmol, 68.83% yield, 98.3% purity, HCl) was obtained as a yellow oil, which was determined by HNMR, LCMS and Melting Point.

TLC (SiO₂, DCM: MeOH=10/1, Rf=0.4) and Melting Point=189.4° C.

LCMS: MS (ESI) Retention time: 0.689 min (M+H)⁺=453.2.

LCMS: MS (ESI) Retention time: 1.573 min (M+H)⁺=453.2.

¹H NMR (400 MHz, DMSO-d₆) δ=10.40 (br s, 1H), 8.11-8.09 (d, J=9.2 Hz, 2H), 7.6-7.64 (d, J=8.8 Hz, 1H), 7.04-7.02 (d, J=8.8 Hz, 3H), 3.92 (s, 3H), 3.48-3.44 (t, J=7.2 Hz, 2H), 3.38-3.35 (d, J=11.6 Hz, 2H), 3.03-2.99 (m, 5H), 2.80-2.78 (m, 2H), 1.76-1.71 (m, 7H), 1.58-1.54 (m, 2H), 1.37 (m, 1H).

### Synthesis of Compound 006-012 (SND 444)

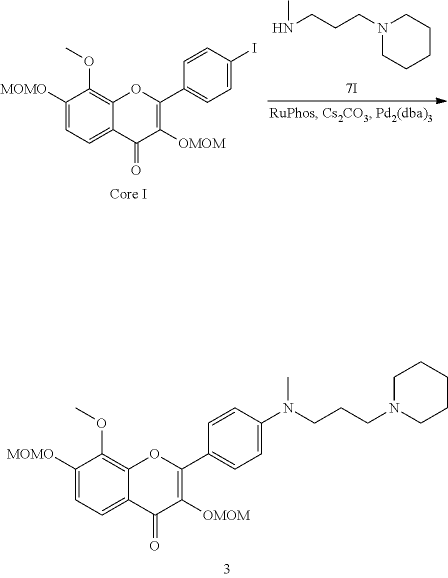

To a solution of Compound 71 (94.09 mg, 602.09 μmol, 5.72 μL, 1.00 eq) and Core I (300 mg, 602.09 μmol, 1.00 eq) in DMF (3 mL) was added RuPhos (56.19 mg, 120.42 μmol, 0.20 eq), Cs₂CO₃ (588.52 mg, 1.81 mmol, 3.00 eq) and Pd₂ (dba)₃ (110.27 mg, 120.42 μmol, 0.20 eq) at 25° C. The mixture was stirred at 100° C. for 12 hr under N₂. LCMS showed Core I consumed. Several new peaks were shown on LCMS and 24.518% of desired compound was detected. The reaction mixture was filtered and concentrated under reduced pressure to give a residue. Without purification. It was put in the next reaction. Compound 3 (300 mg, 569.67 μmol, 94.62% yield) was obtained as a red oil.

LCMS: MS (ESI) Retention time: 0.735 min (M+H)⁺=527.4.

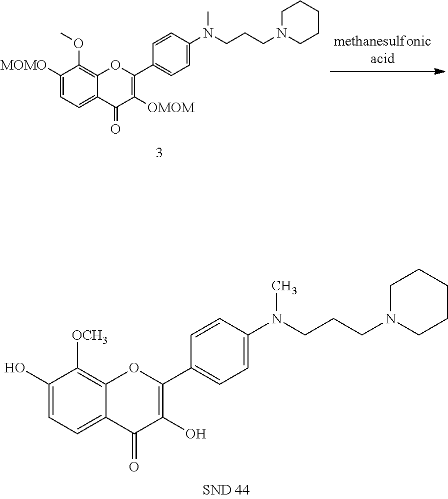

To a solution of Compound 3 (300 mg, 569.67 μmol, 1.00 eq) in MeOH (2.1 mL) and DCM (0.3 mL) was added methanesulfonic acid (54-75 mg, 569.67 μmol, 40.55 μL, 1 eq). The mixture was stirred at 60° C. for 1 hr. LCMS showed Compound 3 was consumed completely and 24.010% of desired mass was detected. The reaction mixture was concentrated under reduced pressure to give a residue. The residue was purified by prep-HPLC (column: 3_Phenomenex Luna C18; 75×30 mm×3 um; mobile phase: [water (HCl) -ACN]; B %: 20%-40%, 6 min). Compound 006-012 (SND 444) (40 mg, 84.21 μmol, 14.78% yield, 100% purity, HCl) was obtained as a yellow oil, which was determined by HNMR and LCMS.

TLC (SiO₂, DCM: MeOH=10/1, Rf=0.1).

LCMS: MS (ESI) Retention time: 0.782 min (M+H)⁺=439.2.

LCMS: MS (ESI) Retention time: 1.189 min (M+H)⁺=439.1.

¹H NMR (400 MHz, DMSO-d₆) δ=8.10-8.08 (d, J=9.2 Hz, 2H), 7.66-7.64 (d, J=8.8 Hz, 1H), 7.02-7.00 (m, 1H), 6.95-6.93 (d, J=8.8 Hz, 2H), 3.92 (s, 3H), 3.50 (m, 2H), 3.40-3.37 (m, 2H), 3.37-3.03 (m, 2H), 3.01 (s, 3H), 2.83-2.81 (m, 2H), 2.02-1.98 (m, 2H), 1.76-1.66 (m, 5), 1.37-1.34 (m, 1H).

### Synthesis of Compound 006-014 (SND 43g)

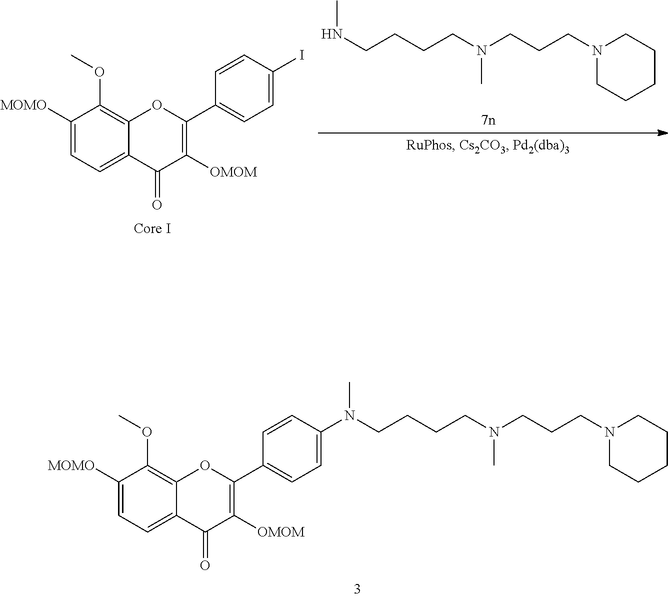

To a solution of Compound 7n (145-35 mg, 602.09 μmol, 1.00 eq) and Core I (300 mg, 602.09 μmol, 1.00 eq) in DMF (5 mL) was added RuPhos (56.19 mg, 120.42 μmol, 0.20 eq), Cs₂CO₃ (588.52 mg, 1.81 mmol, 3.00 eq) and Pd₂ (dba)₃ (110.27 mg, 120.42 μmol, 0.20 eq) at 25° C. The mixture was degassed and purged with N₂ for 3 times, and then the mixture was stirred at 100° C. for 12 hr under N₂ atmosphere. LCMS showed Core I consumed. Several new peaks were shown on LCMS and 15-143% of desired compound was detected. The reaction mixture was filtered and concentrated under reduced pressure to give a residue. Without purification. It was put in the next reaction. Compound 3 (300 mg, 490.38 μmol, 81.45% yield) was obtained as a red oil.

LCMS: MS (ESI) Retention time: 0.688 min (M+H)⁺=612.5.

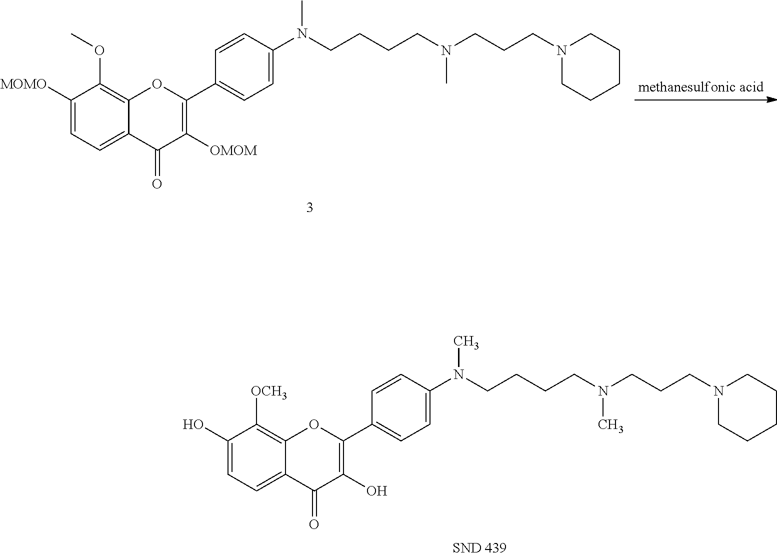

To a solution of Compound 3 (300 mg, 490.38 μmol, 1 eq) in MeOH (2.1 mL) and DCM (0.3 mL) was added methanesulfonic acid (565.54 mg, 5.88 mmol, 418.92 μL, 12 eq). The mixture was stirred at 60° C. for 1 hr. LCMS showed Compound 3 was consumed completely and 19.646% of desired mass was detected. The reaction mixture was concentrated under reduced pressure to give a residue. The residue was purified by prep-HPLC (column: Phenomenex Synergi C18; 150×25 mm×10 um; mobile phase: [water (HCl) -ACN]; B %: 13%-33%, 9 min). Compound 006-014 (SND 439) (55 mg, 94.08 μmol, 19.19% yield, 95.816% purity, HCl) was obtained as a yellow solid, which was determined by HNMR and LCMS.

TLC (SiO₂, DCM: MeOH=10/1, Rf=0.1).

LCMS: MS (ESI) Retention time: 0.732 min (M+H)⁺=524.5.

LCMS: MS (ESI) Retention time: 0.657 min (M+H)⁺=524.5.

¹H NMR (400 MHz, DMSO-d₆) δ=10.83 (br d, J=3.6 Hz, 2H), 8.11-8.09 (d, J=9.2 Hz, 2H), 7.66-7.64 (d, J=8.8 Hz, 1H), 7.05-7.03 (d, J=8.8 Hz, 3H), 3.92 (s, 3H), 3.49-3.46 (t, J=7.2 Hz, 2H), 3.40-3.38 (d, J=11.2 Hz, 2H), 3.27-3.04 (m, 6H), 3.03 (s, 3H), 2.84-2.82 (m, 2H), 2.72-2.71 (m, 3H), 2.20-2.18 (m, 2H), 1.85-1.73 (m, 7H), 1.58 (m, 2H), 1.38 (m, 1H).

### Synthesis of Compound 006-016 (SND 443)

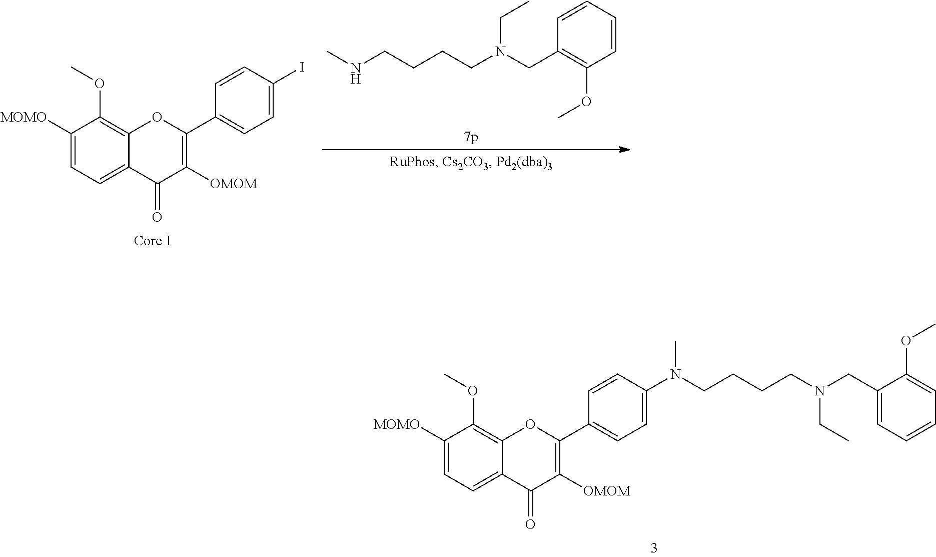

To a solution of Core I (300 mg, 602.09 μmol, 1.00 eq), RuPhos (56.19 mg, 120.42 μmol, 0.20 eq), Pd₂ (dba)₃ (110.27 mg, 120.42 μmol, 0.20 eq) and Cs₂CO₃ (588.52 mg, 1.81 mmol, 3.00 eq) in DMF (3 mL), then added Compound 7P (150.75 mg, 602.09 μmol, 1.00 eq) at 25° C., the mixture was stirred at 100° C. for 16 hr. LCMS showed Core I consumed. Several new peaks were shown on LCMS and 33.183% of desired compound was detected. The reaction mixture was filtered and concentrated under reduced pressure to give a residue. Without purification. It was put in the next reaction. Compound 3 (300 mg, 483.30 μmol, 80.27% yield) was obtained as a red oil.

LCMS: MS (ESI) Retention time: 0.776 min (M+H)⁺=621.4.

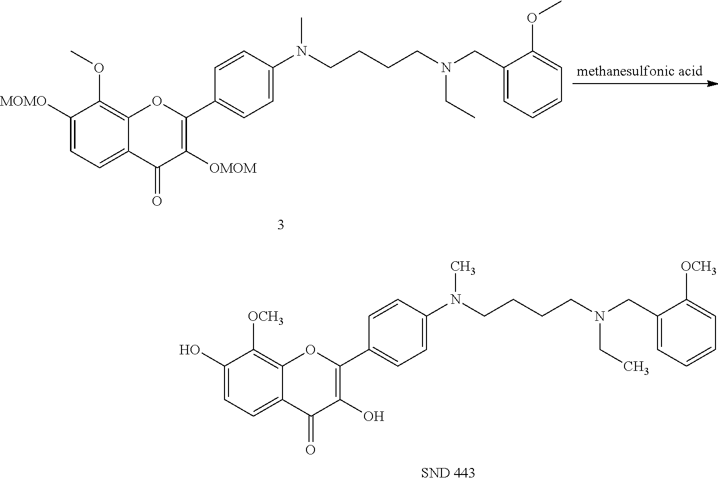

To a solution of Compound 3 (300 mg, 483.30 μmol, 1.00 eq) in MeOH (2.1 mL) and DCM (0.3 mL) was added methanesulfonic acid (557.38 mg, 5.80 mmol, 412.87 μL, 12 eq). The mixture was stirred at 60° C. for 0.5 hr. LCMS showed Compound 3 was consumed completely and 41.981% of desired mass was detected. The reaction mixture was concentrated under reduced pressure to give a residue. The crude product was purified by reversed-phase HPLC (0.1% HCl condition). Compound 006-016 (SND 443) (155 mg, 258.85 μmol, 53.56% yield, 95.036% purity, HCl) was obtained as a yellow oil, which was determined by HNMR and LCMS.

TLC (SiO₂, DCM: MeOH=10/1, Rf=0.1).

LCMS: MS (ESI) Retention time: 0.830 min (M+H)⁺=533.4.

LCMS: MS (ESI) Retention time: 1.755 min (M+H)⁺=533.3.

¹H NMR (400 MHz, DMSO-d₆) δ=9.82 (br s, 1H), 8.11-8.09 (d, J=9.2 Hz, 2H), 7.67-7.64 (d, J=8.8 Hz, 1H), 7.57 (m, 1H), 7.46-7.44 (m, 1H), 7.11-7.09 (d, J=8.4 Hz, 1H), 7.04-6.99 (m, 4H), 4.24-4.22 (t, J=4.4 Hz, 2H), 3.92 (s, 3H), 3.82 (s, 3H), 3.46 (m, 2H), 3.01 (s, 7H), 1.79-1.73 (m, 2H), 1.56-1.53 (m, 2H), 1.28-1.25 (t, J=7.2 Hz, 3H).

### Synthesis of Compound 006-017 (SND 438)

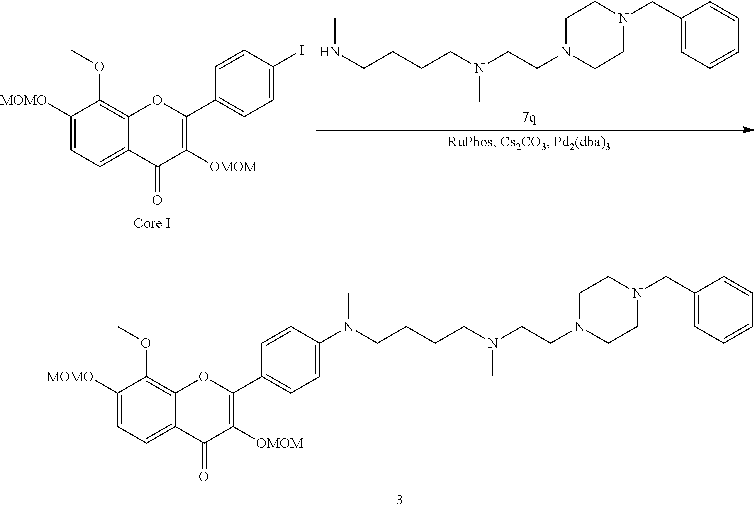

To a solution of Core I (300 mg, 602.09 μmol, 1.00 eq), RuPhos (56.19 mg, 120.42 μmol, 0.20 eq), Pd₂ (dba)₃ (110.27 mg, 120.42 μmol, 0.20 eq) and Cs₂CO₃ (588.52 mg, 1.81 mmol, 3.00 eq) in DMF (5 mL), then added Compound 7q (191.77 mg, 602.09 μmol, 1.00 eq) at 25° C., that mixture was degassed and purged with N₂ for 3 times, the mixture was stirred at 100° C. for 12 hr under N₂ atmosphere. LCMS showed Core I consumed. Several new peaks were shown on LCMS and 44.988% of desired compound was detected. The reaction mixture was filtered and concentrated under reduced pressure to give a residue. Without purification. It was put in the next reaction. Compound 3 (300 mg, 435.51 μmol, 72.33% yield) was obtained as a red oil.

LCMS: MS (ESI) Retention time: 0.686 min (M+H)⁺=689.6.

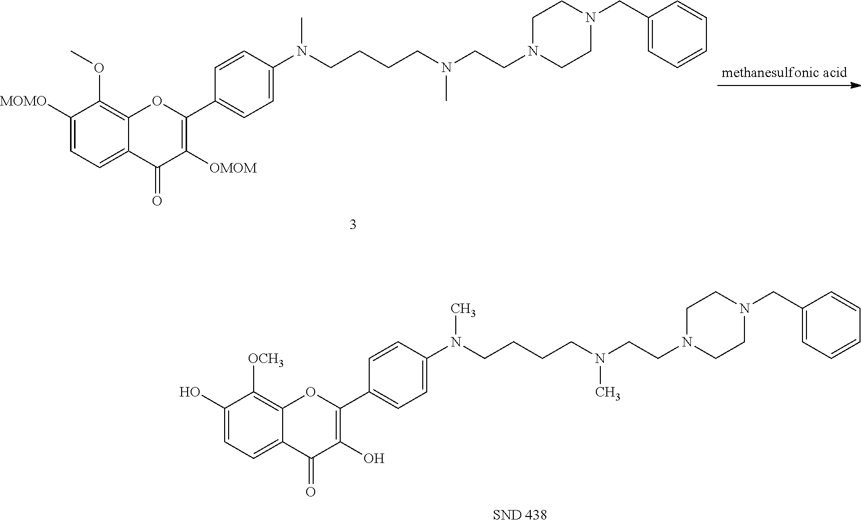

To a solution of Compound 3 (300 mg, 435.51 μmol, 1.00 eq) in MeOH (4.2 mL) and DCM (0.6 mL) was added methanesulfonic acid (502.26 mg, 5.23 mmol, 372.04 μL, 12 eq). The mixture was stirred at 60° C. for 1 hr. LCMS showed Compound 3 was consumed completely and 48.006% of desired mass was detected. The reaction mixture was concentrated under reduced pressure to give a residue. The crude product was purified by reversed-phase HPLC (0.1% HCl condition). Compound 006-017 (SND 438) (215 mg, 310.89 μmol, 71.39% yield, 92.140% purity, HCl) was obtained as a yellow solid, which was determined by HNMR and LCMS.

TLC (SiO₂, DCM: MeOH=10/1, Rf=0.2).

LCMS: MS (ESI) Retention time: 0.676 min (M+H)⁺=601.4.

LCMS: MS (ESI) Retention time: 1.507 min (M+H)⁺=601.4.

¹H NMR (400 MHz, DMSO-d₆) δ=10.74 (br s, 1H), 8.10-8.08 (d, J=9.2 Hz, 2H), 7.67-7.64 (m, 3H), 7.46-7.44 (m, 3H), 7.03-7.00 (d, J=8.8 Hz, 1H), 6.96 (m, 2H), 4.39 (s, 2H), 3.92 (s, 3H), 3.66-3.59 (m, 2H), 3.46 (m, 8H), 3.36-3.32 (m, 4H), 3.14 (m, 2H), 3.02 (s, 3H), 2.78 (s, 3H), 1.75-1.73 (m, 2H), 1.59-1.57 (m, 2H).

### Synthesis of Compound 006-018 (SND 437)

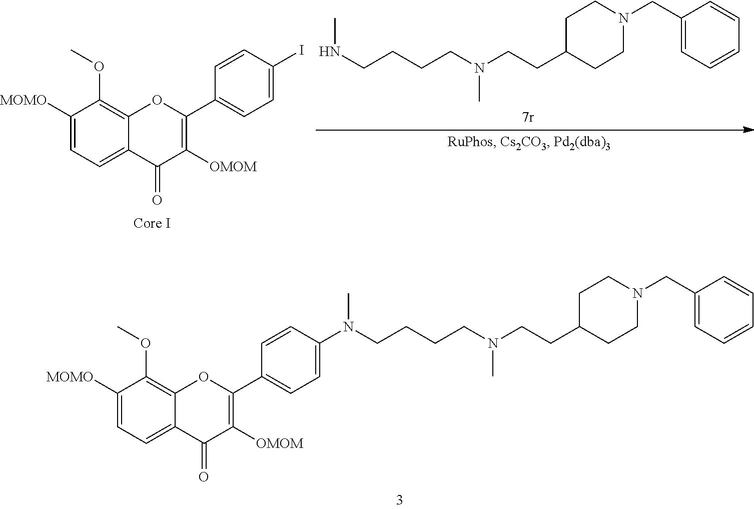

To a solution of Core I (300 mg, 602.09 μmol, 1.00 eq), RuPhos (56.19 mg, 120.42 μmol, 0.20 eq), Pd₂ (dba)₃ (110.27 mg, 120.42 μmol, 0.20 eq) and Cs₂CO₃ (588.52 mg, 1.81 mmol, 3.00 eq) in DMF (5 mL), then added Compound 7r (191.17 mg, 602.09 μmol, 1.00 eq) at 25° C., that mixture was degassed and purged with N₂ for 3 times, the mixture was stirred at 100° C. for 12 hr under N₂ atmosphere. LCMS showed Core I consumed. Several new peaks were shown on LCMS and 41.616% of desired compound was detected. The reaction mixture was filtered and concentrated under reduced pressure to give a residue. Without purification. It was put in the next reaction.

Compound 3 (300 mg, 436.13 μmol, 72.44% yield) was obtained as a red oil.

LCMS: MS (ESI) Retention time: 0.796 min (M+H)⁺=688.6.

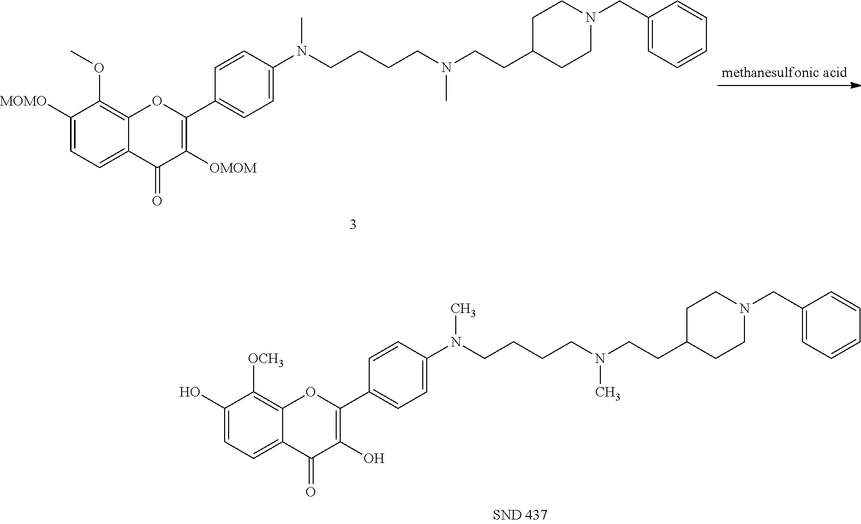

To a solution of Compound 3 (299.57 mg, 435.51 μmol, 1.00 eq) in MeOH (4.2 mL) and DCM (0.6 mL) was added methanesulfonic acid (502.26 mg, 5.23 mmol, 372.04 μL, 12 eq). The mixture was stirred at 60° C. for 1 hr. LCMS showed Compound 3 was consumed completely and 51.445% of desired mass was detected. The reaction mixture was concentrated under reduced pressure to give a residue. The crude product was purified by reversed-phase HPLC (0.1% HCl condition). Compound 006-018 (SND 437) (190 mg, 289.56 μmol, 66.49% yield, 96.960% purity, HCl) was obtained as a yellow solid, which was determined by HNMR and LCMS.

TLC (SiO₂, DCM: MeOH=10/1, Rf=0.2).

LCMS: MS (ESI) Retention time: 0.686 min (M+H)⁺=600-5.

LCMS: MS (ESI) Retention time: 1.523 min (M+H)⁺=600.4.

¹H NMR (400 MHz, DMSO-d₆) δ=11.03 (br s, 1H), 10.62 (br s, 1H), 8.10-8.08 (d, J=9.2 Hz, 2H), 7.66-7.62 (m, 3H), 7.44-7.42 (m, 3H), 7.04-7.01 (m, 3H), 4.23-4.21 (d, J=5.2 Hz, 2H), 3.92 (s, 3H), 3.70 (m, 2H), 3.47-3.45 (m, 2H), 3.28-3.25 (m, 2H), 3.06-2.98 (m, 8H), 2.89-2.75 (m, 2H), 2.67-2.66 (m, 3H), 1.81-1.72 (m, 4H), 1.60-1.55 (m, 7H).

### Synthesis of Compound 006-011 (SND 44s)

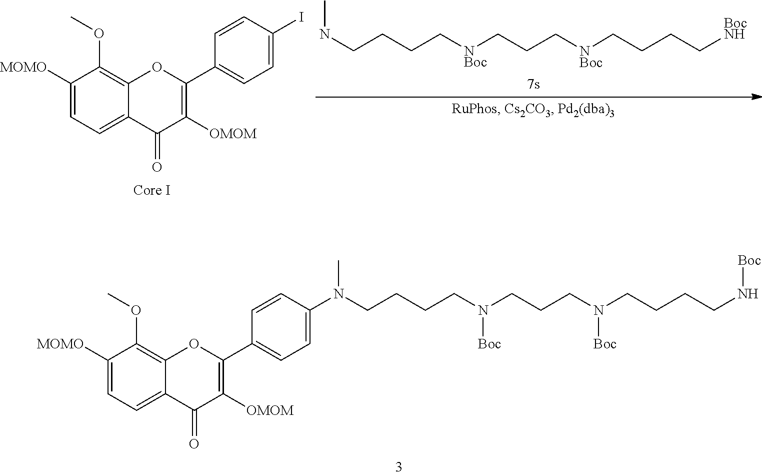

To a solution of Compound 7s (200 mg, 401.39 μmol, 1.00 eq), RuPhos (37.46 mg, 80.28 μmol, 0.20 eq), Pd₂ (dba)₃ (73.51 mg, 80.28 μmol, 0.20 eq) and Cs₂CO₃ (392.35 mg, 1.20 mmol, 3.00 eq) in DMF (1 mL), then added Core I (213.04 mg, 401.39 μmol, 1.00 eq) at 25° C., the mixture was stirred at 100° C. for 16 hr. LCMS showed Core I was consumed. Several new peaks were shown on LCMS and 47.340% of desired compound was detected. The reaction mixture was filtered and concentrated under reduced pressure to give a residue. Without purification. It was put in the next reaction. Compound 3 (300 mg, 332.93 μmol, 82.94% yield) was obtained as a red oil.

LCMS: MS (ESI) Retention time: 1.157 min (M+H)⁺=901.7.

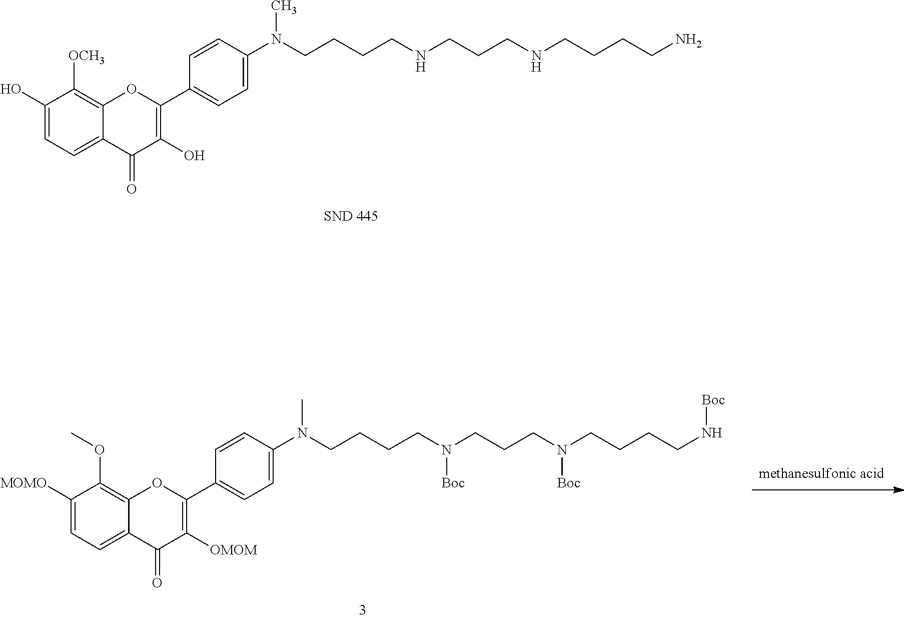

To a solution of Compound 3 (300 mg, 332.93 μmol, 1.00 eq) in MeOH (2.1 mL) and DCM (0.3 mL) was added methanesulfonic acid (383.96 mg, 4.00 mmol, 284.41 μL, 12 eq). The mixture was stirred at 60° C. for 1 hr. LCMS showed Compound 3 was consumed completely and 47.286% of desired mass was detected. The reaction mixture was concentrated under reduced pressure to give a residue. The crude product was purified by reversed-phase HPLC (0.1% HCl condition). Compound 006-019 (SND 445) (135 mg, 245.86 μmol, 73.85% yield, 100% purity, HCl) was obtained as a yellow oil, which was determined by HNMR and LCMS.

TLC (SiO₂, DCM: MeOH=10/1, Rf=0.05).

LCMS: MS (ESI) Retention time: 0.710 min (M+H)⁺=513.4.

LCMS: MS (ESI) Retention time: 0.962 min (M+H)⁺=513.2.

¹H NMR (400 MHz, DMSO-d₆) δ=8.10-8.08 (d, J=8.8 Hz, 2H), 7.66-7.64 (d, J=8.8 Hz, 1H), 7.07-7.06 (m, 2H), 7.01-6.99 (d, J=8.8 Hz, 1H), 3.90 (s, 3H), 3.48-3.46 (m, 2H), 3.02 (s, 3H), 2.99-2.96 (t, J=7.6 Hz, 4H), 2.9-2.89 (m, 4H), 2.79 (m, 2H), 2.02-1.99 (m, 2H), 1.64-1.60 (m, 8H).

### Synthesis of Compound 006-020 (SND 446)

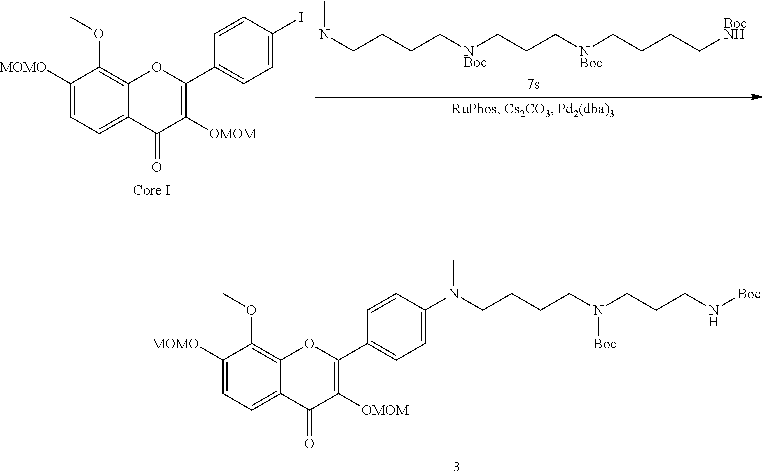

To a solution of Compound 7s (208.01 mg, 602.09 μmol, 1.00 eq) and Core I (300 mg, 602.09 μmol, 1.00 eq) in DMF (3 mL) was added RuPhos (56.19 mg, 120.42 μmol, 0.20 eq), Cs₂CO₃ (588.52 mg, 1.81 mmol, 3.00 eq) and Pd₂ (dba)₃ (110.27 mg, 120.42 μmol, 0.20 eq) at 25° C. The mixture was stirred at 100° C. for 12 hr under N₂. LCMS showed Core I was consumed completely and 40.463% of desired mass was detected. The reaction mixture was filtered and concentrated under reduced pressure to give a residue. Without purification. It was put in the next reaction. Compound 3 (300 mg, 419.10 μmol, 69.61% yield) was obtained as a red oil.

LCMS: MS (ESI) Retention time: 1.056 min (M+H)⁺=716.5.

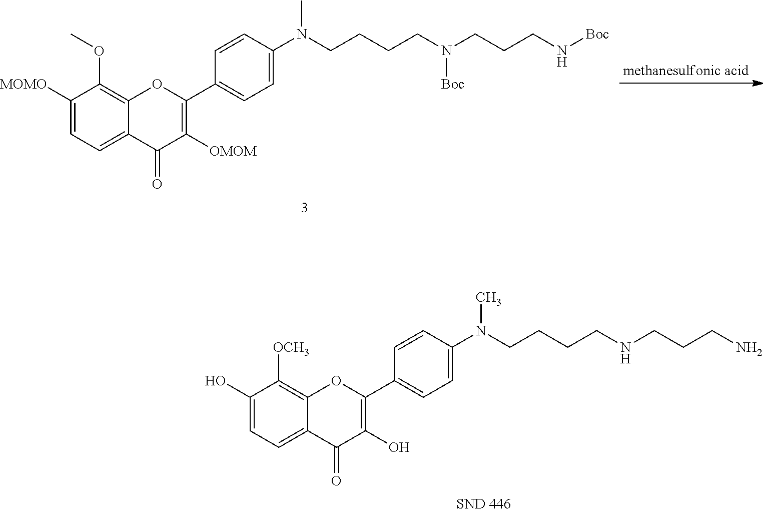

To a solution of Compound 3 (300 mg, 419.10 μmol, 1.00 eq) in MeOH (2.1 mL) and DCM (0.3 mL) was added methanesulfonic acid (483.33 mg, 5.03 mmol, 358.02 μL, 12 eq). The mixture was stirred at 60° C. for 1 hr. LCMS showed Compound 3 was consumed completely and 28.727% of desired mass was detected. The reaction mixture was concentrated under reduced pressure to give a residue. The crude product was purified by reversed-phase HPLC (0.1% HCl condition). Compound 006-020 (SND 446) (105 mg, 216.61 μmol, 51.69% yield, 95.712% purity, HCl) was obtained as a yellow solid, which was determined by HNMR and LCMS.

TLC (SiO₂, DCM: MeOH=10/1, Rf=0.05).

LCMS: MS (ESI) Retention time: 0.699 min (M+H)⁺=428.3.

LCMS: MS (ESI) Retention time: 1.283 min (M+H)⁺=428.2.

¹H NMR (400 MHz, DMSO-d₆) δ=8.02-7.80 (d, J=8.8 Hz, 2H), 7.67-7.65 (d, J=8.8 Hz, 1H), 7.00-6.98 (d, J=8.8 Hz, 1H), 6.83-6.80 (d, J=8.8 Hz, 2H), 3.91 (s, 3H), 3.17-3.15 (m, 2H), 2.99-2.86 (m, 6H), 1.94-1.90 (m, 2H), 1.71-1.63 (m, 4H).

### Synthesis of Compound 006-021 (SND 44)

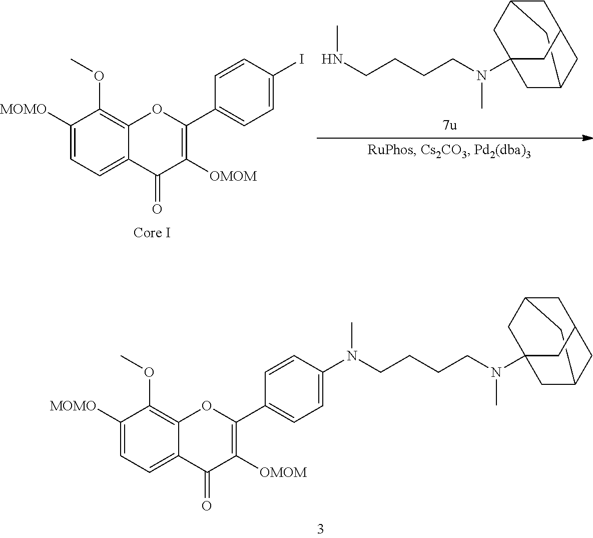

To a solution of Core I (300 mg, 619.53 μmol, 1.00 eq), RuPhos (57.82 mg, 123.91 μmol, 0.20 eq), Pd₂ (dba)₃ (113.46 mg, 123.91 μmol, 0.20 eq) and Cs₂CO₃ (605.57 mg, 1.86 mmol, 3.00 eq) in DMF (1 mL), then added Compound 7n (155.14 mg, 619.53 μmol, 1.00 eq) at 25° C., the mixture was stirred at 100° C. for 16 hr under N₂. LCMS showed Core I consumed. Several new peaks were shown on LCMS and 44.365% of desired compound was detected. The reaction mixture was filtered and concentrated under reduced pressure to give a residue. Without purification. It was put in the next reaction. Compound 3 (300 mg, 483.27 μmol, 78.01% yield) was obtained as a red oil.

LCMS: MS (ESI) Retention time: 0.861 min (M+H)⁺=621.5.

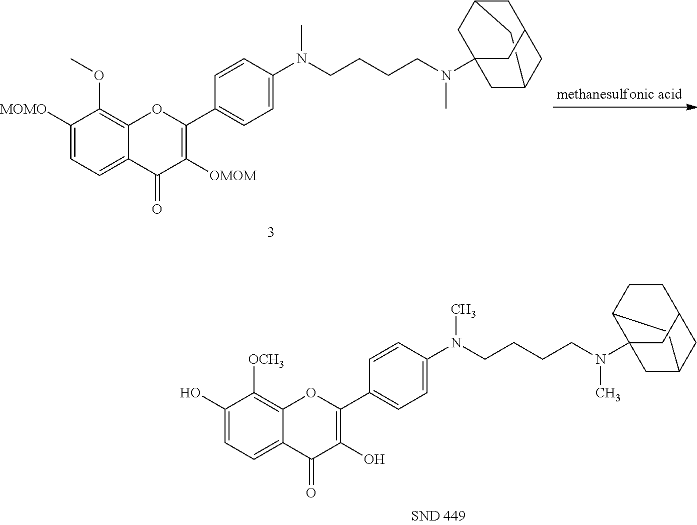

To a solution of Compound 3 (300 mg, 483.27 μmol, 1.00 eq) in MeOH (1.4 mL) and HCl (0.2 mL) was added methanesulfonic acid (557-34 mg, 5.80 mmol, 412.84 μL, 12 eq). The mixture was stirred at 60° C. for 1 hr. LCMS showed Compound 3 was consumed completely and 42.243% of desired mass was detected. The reaction mixture was concentrated under reduced pressure to give a residue. The crude product was purified by reversed-phase HPLC (0.1% HCl condition). Compound 006-021 (SND 449) (145 mg, 254.77 μmol, 52.72% yield, 100% purity, HCl) was obtained as a yellow solid, which was determined by HNMR and LCMS.

TLC (SiO₂, DCM: MeOH=10/1, Rf=0.05).

LCMS: MS (ESI) Retention time: 0.760 min (M+H)⁺=533.4.

LCMS: MS (ESI) Retention time: 1.798 min (M+H)⁺=533.4.

¹H NMR (400 MHz, DMSO-d₆) δ=9.79 (br s, 1H), 8.11-8.08 (d, J=8.8 Hz, 2H), 7.67-7.64 (d, J=8.8 Hz, 1H), 7.03-6.97 (m, 3H), 3.92 (s, 3H), 3.49-3.44 (m, 2H), 3.40-3.32 (m, 1H), 3.02 (s, 3H), 2.74-2.66 (m, 1H), 2.62-2.61 (m, 3H), 2.13 (s, 3H), 1.99-1.96 (m, 3H), 1.91-1.88 (m, 3H), 1.75-1.72 (m, 3H), 1.61 (m, 7H).

### Synthesis of Compound 007-004 (SND 411)

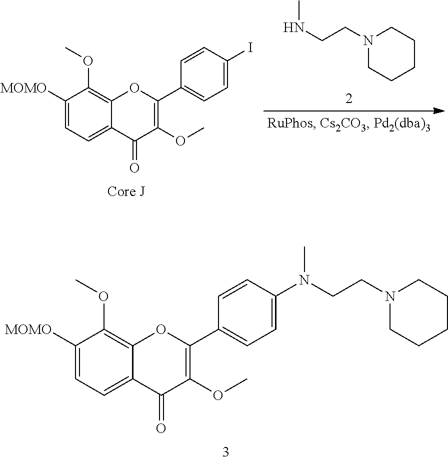

To a solution of Core J (300 mg, 640.70 umol, 1.00 eq), Compound 2 (114.49 mg, 640.70 umol, 1.00 eq, HCl) in DMF (3 mL) was added Cs₂CO₃ (626.26 mg, 1.92 mmol, 3.00 eq), RuPhos (29.90 mg, 64.07 umol, 0.10 eq), Pd₂(dba)₃ (58.67 mg, 64.07 umol, 0.1 eq), and the mixture was stirred at 100° C. for 16 h under N₂. LCMS showed Core J was consumed and desired mass was detected. The reaction mixture was filtered and concentrated under reduced pressure to give a residue. Compound 3 (320 mg, crude) was obtained as a red oil.

LCMS: MS (ESI) Retention time: 0.721 min, (M+1)⁺=483.3.

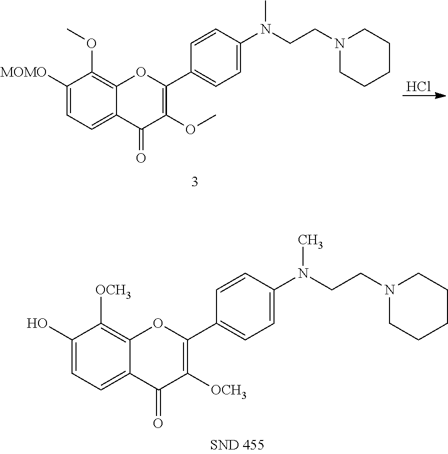

To a solution of Compound 3 (320 mg, 663.12 umol, 1.00 eq) in dioxane (2 mL) was added HCl/dioxane (4 M, 2 mL, 12.06 eq), and the mixture was stirred at 25° C. for 1 h. LCMS showed Compound 3 was consumed and desired mass was detected. The reaction mixture was concentrated under pressure to give a residue. The crude product was purified by reversed-phase HPLC (0.1% HCl condition). Compound 007-004 (SND 455) (220 mg, 453.92 umol, 68.45% yield, 98% purity, HCl) was obtained as an orange solid, which was confirmed by LCMS and HNMR.

LCMS: MS (ESI) Retention time: 0.682 min, (M+1)⁺=439.3.

LCMS: MS (ESI) Retention time: 1.465 min, (M+1)⁺=425.1.

¹H NMR (400 MHz, DMSO-d6) δ=7.99-7.97 (d, J=8.8 Hz, 2H), 7.64-7.62 (d, J=8.8 Hz, 1H), 7.01-6.96 (m, 3H), 3.90 (s, 3H), 3.87-3.85 (m, 2H), 3.75 (s, 3H), 3.50-3.48 (d, J=11.6 hZ, 2H), 3.21-3.17 (m, 2H), 3.03 (s, 3H), 2.97-2.92 (m, 2H), 1.83-1.69 (m, 5H), 1.40-1.37 (m, 1H).

TLC (SiO2, DCM/MeOH=10/1, Rf=0.5).

### Synthesis of Compound 007-00s (SND 450)

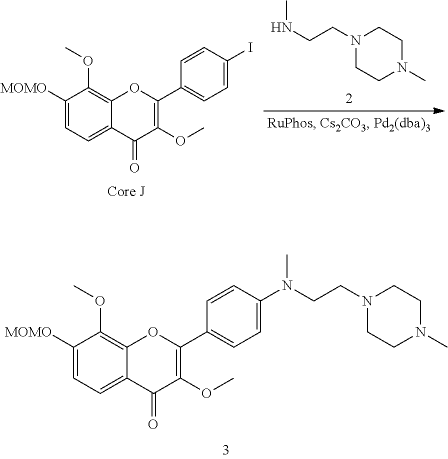

To a solution of Core J (300 mg, 640.70 umol, 1.00 eq), Compound 2 (100.75 mg, 640.70 umol, 1.00 eq) in DMF (4 mL) was added Cs₂CO₃ (626.26 mg, 1.92 mmol, 3.00 eq), RuPhos (29.90 mg, 64.07 umol, 0.10 eq), Pd₂(dba)₃ (58.67 mg, 64.07 umol, 0.10 eq), and the mixture was stirred at 100° C. for 16 h under N₂. LCMS showed Core J was consumed and 67% of desired mass was detected. The reaction mixture was filtered and concentrated under reduced pressure to give a residue. Compound 3 (320 mg, crude) was obtained as a red oil.

LCMS MS (ESI) Retention time: 0.689 min, (M+1)⁺=498.2.

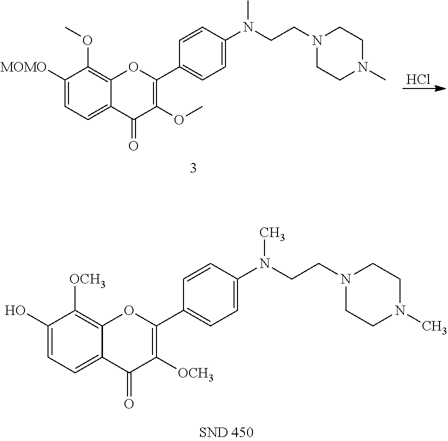

To a solution of Compound 3 (320 mg, 643.11 umol, 1.00 eq) in dioxane (2 mL) was added HCl/dioxane (4 M, 2 mL, 12.4 eq), and the mixture was stirred at 25° C. for 2 h. LCMS showed Compound 3 was consumed and desired mass was detected. The reaction mixture was concentrated under reduced pressure to give a residue. The crude product was purified by reversed-phase HPLC (0.1% HCl condition). Compound 007-005 (SND 450) (280 mg, 560.01 umol, 87.08% yield, 98% purity, HCl) was obtained as a red solid, which was confirmed by HNMR and LCMS.

LCMS: MS (ESI) Retention time: 0.658 min, (M+1)⁺=454.2.

LCMS: MS (ESI) Retention time: 1.390 min, (M+1)⁺=454.2.

¹H NMR (400 MHz, DMSO-d6) δ=12.12-12.06 (br s, 1H), 8.00-7.97 (d, J=9.2 Hz, 2H), 7.64-7.62 (d, J=8.8 Hz, 1H), 7.04-7.01 (m, 3H), 3.96-3.92 (m, 2H), 3.91 (s, 3H), 3.81 (m, 2H), 3.77 (s, 3H), 3.72-3.70 (m, 2H), 3.51 (s, 4H), 3.37 (s, 2H), 3.06 (s, 3H), 2.84 (s, 3H).

TLC (SiO2, DCM/MeOH=10/1, Rf=0.5.

### Synthesis of Compound 007-006 (SND 456)

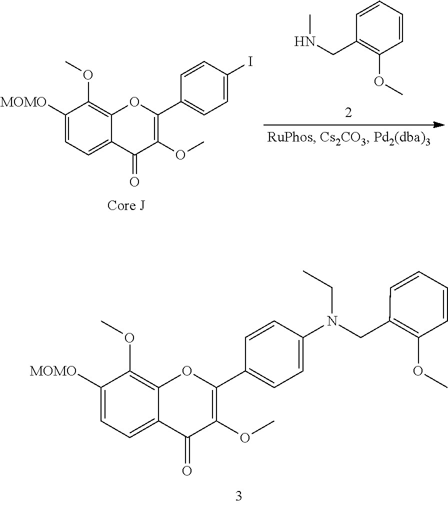

To a solution of Core J (300 mg, 640.70 umol, 1.00 eq), Compound 2 (129.22 mg, 640.70 umol, 1.00 eq, HCl) in DMF (3 mL) was added Cs₂CO₃ (626.26 mg, 1.92 mmol, 3.00 eq), RuPhos (29.90 mg, 64.07 umol, 0.10 eq), Pd₂(dba)₃ (58.67 mg, 64.07 umol, 0.10 eq), and the mixture was stirred at 100° C. for 16 h under N₂. LCMS showed Core J was consumed and desired mass was detected. The reaction mixture was filtered and concentrated under pressure to give a residue. The crude product was purified by reversed-phase HPLC (0.1% FA condition). Compound 3 (180 mg, crude) was obtained as a yellow oil.

LCMS MS (ESI) Retention time: 0.972 min, (M+1)⁺=506.3.

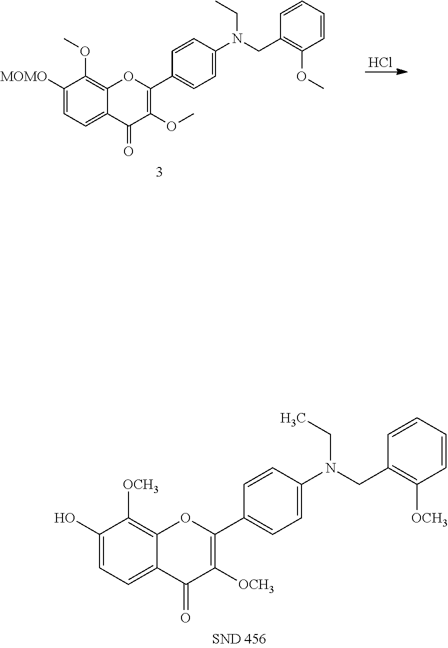

To a solution of Compound 3 (180 mg, 356.04 umol, 1.00 eq) in dioxane (2 mL) was added HCl/dioxane (4 M, 2 mL, 22.47 eq), and the mixture was stirred at 25° C. for 1 h. LCMS showed Compound 3 was consumed and desired mass was detected. The reaction mixture was concentrated under reduced pressure to give a residue. The residue was purified by prep-HPLC (column: 3_Phenomenex Luna C18; 75×30 mm×3 um; mobile phase: [water (HCl)-ACN]; B %: 45%-65%, 8 min). Compound 007-006 (SND 456) (105 mg, 206.64 umol, 58.04% yield, 98% purity, HCl) was obtained as a yellow solid, which was confirmed by LCM and HNMR.

LCMS: MS (ESI) Retention time: 0.896 min, (M+1)⁺=462.2.

LCMS: MS (ESI) Retention time: 2.305 min, (M+1)⁺=462.2.

¹H NMR (400 MHz, DMSO-d6) δ=7.94-7.92 (d, J=9.2 Hz, 2H), 7.63-7.60 (d, J=8.8 Hz, 1H), 7.25-7.23 (m, 1H), 7.05-7.00 (m, 3H), 6.98-6.95 (t, J=7.6 Hz, 1H), 6.87-6.77 (m, 2H), 4.56 (s, 3H), 3.89 (s, 2H), 3.86 (s, 3H), 3.76 (s, 3H), 3.55-3.54 (m, 2H), 1.21-1.17 (m, 3H).

TLC (SiO₂, DCM/MeOH=10/1, Rf=0.3).

### Synthesis of Compound 007-007 (SND 454)

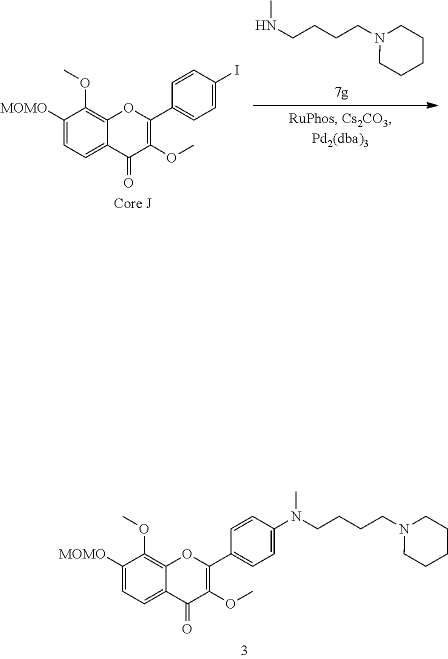

To a solution of Compound 7g (109.11 mg, 640.70 μmol, 5.72 μL, 1.00 eq) and Core J (300 mg, 640.70 μmol, 1.00 eq) in DMF (1.00 mL) was added RuPhos (59.80 mg, 128.14 μmol, 0.20 eq), Cs₂CO₃ (626.26 mg, 1.92 mmol, 3.00 eq) and Pd₂ (dba)₃ (117.34 mg, 128.14 μmol, 0.20 eq) at 25° C. The mixture was stirred at 100° C. for 12 hr under N₂. LCMS showed Core J was consumed. Several new peaks were shown on LCMS and 65.733% of desired compound was detected. The reaction mixture was filtered and concentrated under reduced pressure to give a residue. Without purification. It was put in the next reaction. Compound 3 (300 mg, 587.52 μmol, 91.70% yield) was obtained as a red oil.

LCMS: MS (ESI) Retention time: 0.819 min (M+H)⁺=511.4.

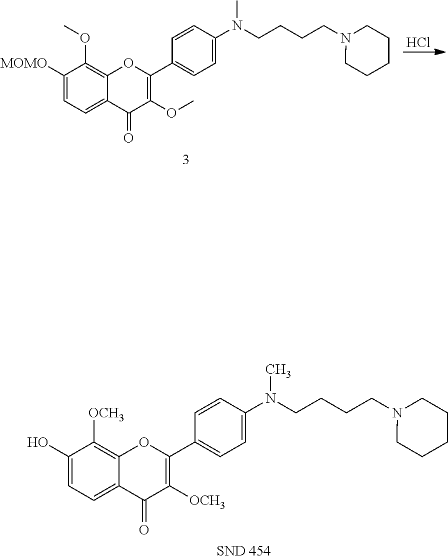

To a solution of Compound 3 (300 mg, 587.52 μmol, 1.00 eq) in HCl/dioxane (8 mL). The mixture was stirred at 25° C. for 1 hr. LCMS showed Compound 3 consumed. Several new peaks were shown on LCMS and 78.683% of desired compound was detected. The reaction mixture was concentrated under reduced pressure to give a residue. The crude product was purified by reversed-phase HPLC (0.1% HCl condition). Compound 007-007 (SND 454) (265 mg, 519.22 μmol, 88.37% yield, 98.559% purity, HCl) was obtained as a yellow solid, which was determined by HNMR and LCMS.

TLC (SiO₂, DCM: MeOH=10/1, Rf=0.2).

LCMS: MS (ESI) Retention time: 0.773 min (M+H)⁺=467.4.

LCMS: MS (ESI) Retention time: 1.575 min (M+H)⁺=467.3.

¹H NMR (400 MHz, DMSO-d₆) δ=8.00-7.97 (d, J=9.2 Hz, 2H), 7.63-7.61 (d, J=8.8 Hz, 1H), 7.03-7.01 (d, J=8.8 Hz, 1H), 6.93-6.91 (d, J=8.8 Hz, 2H), 3.91 (s, 3H), 3.77 (s, 3H), 3.48-3.45 (t, J=7.2 Hz, 2H), 3.39-3.36 (d, J=11.6 Hz, 2H), 3.03-2.99 (m, 5H), 2.82-2.80 (m, 2H), 1.80-1.75 (m, 7H), 1.71-1.67 (m, 2H), 1.59-1.57 (m, 1H).

### Synthesis of Compound 007-012 (SND 451)

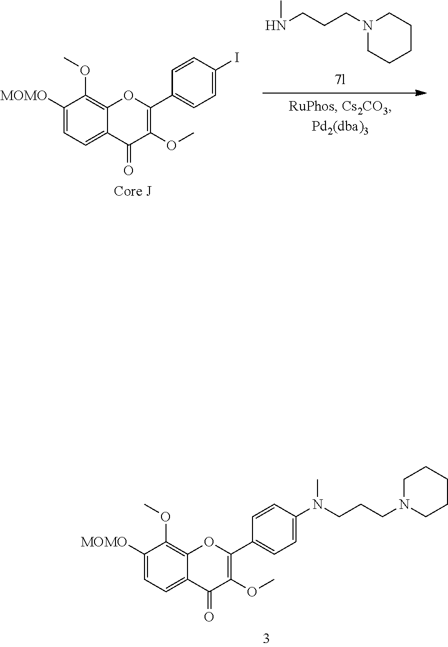

To a solution of Compound 71 (100.12 mg, 640.70 μmol, 5.72 μL, 1.00 eq) and Core J (300 mg, 640.70 μmol, 1.00 eq) in DMF (5 mL) was added RuPhos (59.80 mg, 128.14 μmol, 0.20 eq), Cs₂CO₃ (626.26 mg, 1.92 mmol, 3.00 eq) and Pd₂ (dba)₃ (117.34 mg, 128.14 μmol, 0.20 eq) at 25° C., that mixture was degassed and purged with N₂ for 3 times. The mixture was stirred at 100° C. for 12 hrs under N₂ atmosphere. LCMS showed Core J was consumed. Several new peaks were shown on LCMS and 27.235% of desired compound was detected. The reaction mixture was filtered and concentrated under reduced pressure to give a residue. Without purification. It was put in the next reaction. Compound 3 (300 mg, 604.12 μmol, 94.29% yield) was obtained as a red oil.

LCMS: MS (ESI) Retention time: 0.805 min (M+H)⁺=497.3.

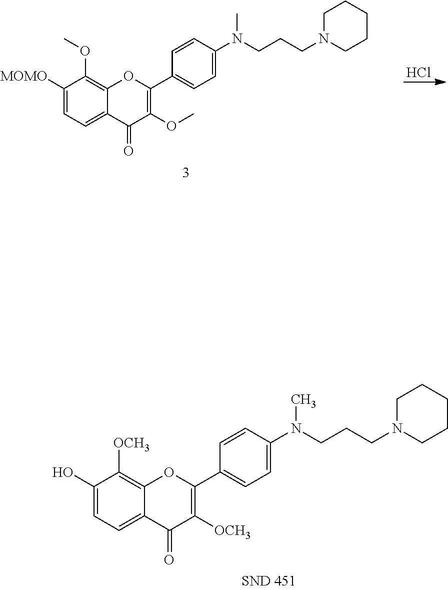

To a solution of Compound 3 (300 mg, 604.12 μmol, 1.00 eq) in HCl/dioxane (3 mL). The mixture was stirred at 25° C. for 1 hr. LCMS showed Compound 3 was consumed completely and 38.776% of desired mass was detected. The reaction mixture was concentrated under reduced pressure to give a residue. The crude product was purified by reversed-phase HPLC (0.1% HCl condition). Compound 007-012 (SND 451) (100 mg, 202.45 μmol, 33.51% yield, 98.997% purity, HCl) was obtained as a yellow oil, which was determined by HNMR and LCMS.

TLC (SiO₂, DCM: MeOH=10/1, Rf=0.3).

LCMS: MS (ESI) Retention time: 0.763 min (M+H)⁺=453.3.

LCMS: MS (ESI) Retention time: 1.176 min (M+H)⁺=453.1.

¹H NMR (400 MHz, METHANOL-d₄) δ=8.25-8.23 (d, J=9.2 Hz, 2H), 7.79-7.77 (d, J=8.8 Hz, 1H), 7.35-7.33 (d, J=8.8 Hz, 2H), 7.03-7.01 (d, J=8.8 Hz, 1H), 4.01 (s, 3H), 3.82 (s, 3H), 3.70-3.67 (t, J=7.5 Hz, 2H), 3.56-3.53 (d, J=11.6 Hz, 2H), 3.23-3.20 (m, 5H), 2.98-2.95 (m, 2H), 2.15-2.11 (m, 2H), 1.92 (m, 2H), 1.81 (m, 3H), 1.59-1.46 (m, 1H).

### Synthesis of Compound 007-014 (SND 419)

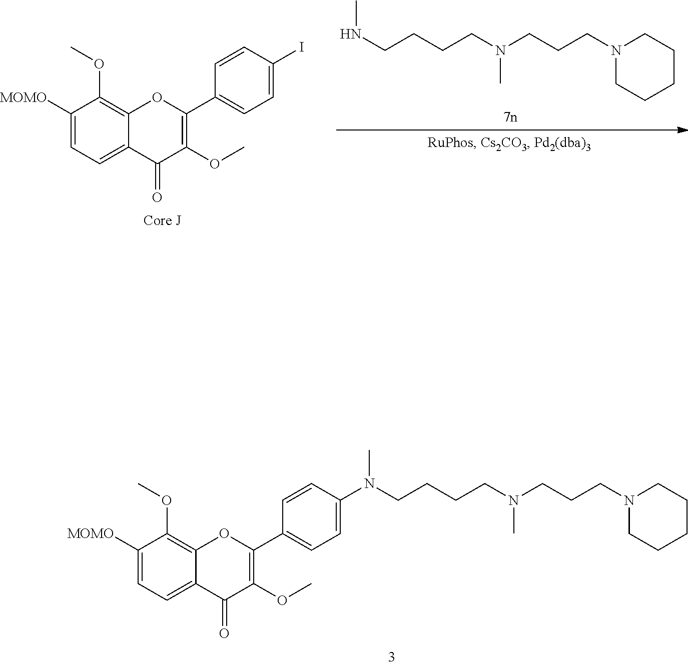

To a solution of Compound 7n (154.67 mg, 640.70 μmol, 1.00 eq) and Core J (300 mg, 640.70 μmol, 1.00 eq) in DMF (5 mL) was added RuPhos (59.80 mg, 128.14 μmol, 0.20 eq), Cs₂CO₃ (626.26 mg, 1.92 mmol, 3.00 eq) and Pd₂ (dba)₃ (117.34 mg, 128.14 μmol, 0.20 eq) at 25° C., that mixture was degassed and purged with N₂ for 3 times, the mixture was stirred at 100° C. for 12 hrs under N₂ atmosphere. LCMS showed Core J consumed. Several new peaks were shown on LCMS and 20.164% of desired compound was detected. The reaction mixture was filtered and concentrated under reduced pressure to give a residue. Without purification. It was put in the next reaction. Compound 3 (300 mg, 515.69 μmol, 80.49% yield) was obtained as a red oil.

LCMS: MS (ESI) Retention time: 0.688 min (M+H)⁺=582.4.

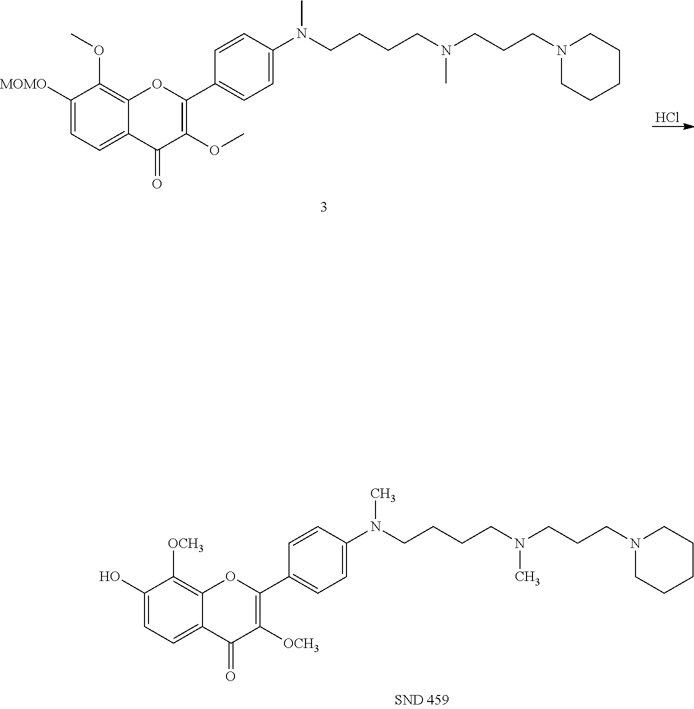

To a solution of Compound 3 (300 mg, 515.69 μmol, 1.00 eq) in HCl/dioxane (5 mL). The mixture was stirred at 25° C. for 1 hr. LCMS showed Compound 3 was consumed completely and 22.223% of desired mass was detected. The reaction mixture was concentrated under reduced pressure to give a residue. The residue was purified by prep-HPLC (column: 3_Phenomenex Luna C18; 75×30 mm×3 um; mobile phase: [water (HCl) -ACN]; B %: 9%-29%, 8 min). Compound 007-014 (SND 459) (90 mg, 156.75 μmol, 30.40% yield, 100% purity, HCl) was obtained as a yellow oil, which was determined by HNMR and LCMS.

TLC (SiO₂, DCM: MeOH=10/1, Rf=0.1).

LCMS: MS (ESI) Retention time: 0.709 min (M+H)⁺=538.4.

LCMS: MS (ESI) Retention time: 1.031 min (M+H)⁺=538.2.

¹H NMR (400 MHz, DMSO-d₆) δ=10.75 (br s, 2H), 8.00-7.97 (d, J=9.2 Hz, 2H), 7.63-7.61 (d, J=8.8 Hz, 1H), 7.03-7.01 (d, J=8.8 Hz, 1H), 6.93-6.91 (d, J=9.2 Hz, 2H), 3.91 (s, 3H), 3.77 (s, 3H), 3.50-3.47 (m, 4H), 3.16-3.07 (m, 6H), 3.03 (s, 3H), 2.85 (m, 2H), 2.74-2.72 (d, J=4.8 Hz, 3H), 2.54 (s, 3H), 2.19 (m, 2H), 1.78-1.60 (m, 10H).

### Synthesis of Compound 007-016 (SND 453)

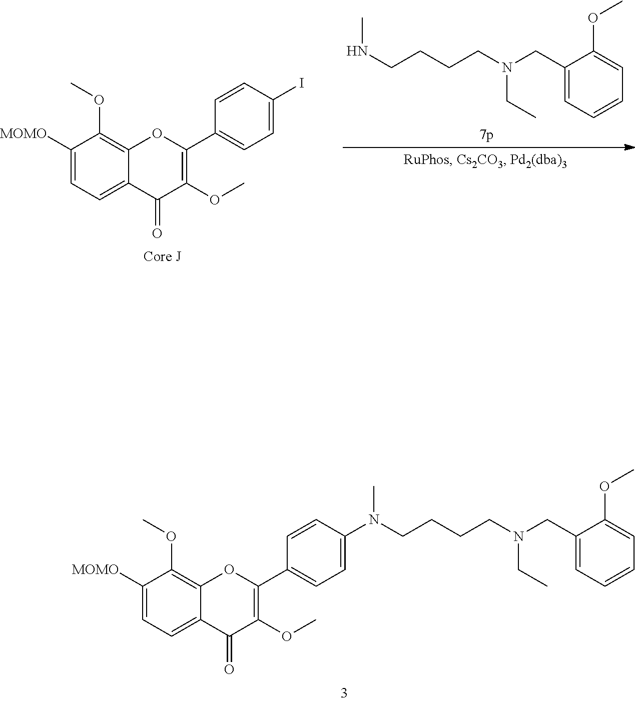

To a solution of Core J (300 mg, 640.70 μmol, 1.00 eq), RuPhos (59.80 mg, 128.14 μmol, 0.20 eq), Pd₂ (dba)₃ (117.34 mg, 128.14 μmol, 0.20 eq) and Cs₂CO₃ (626.26 mg, 1.92 mmol, 3.00 eq) in DMF (5 mL), then added Compound 7p (159.15 mg, 640.70 μmol, 1.00 eq) at 25° C., that mixture was degassed and purged with N₂ for 3 times, the mixture was stirred at 100° C. for 16 hr under N₂ atmosphere. LCMS showed Core J was consumed. Several new peaks were shown on LCMS and 61.559% of desired compound was detected. The reaction mixture was filtered and concentrated under reduced pressure to give a residue. Without purification. It was put in the next reaction. Compound 3 (300 mg, 507.87 μmol, 79.27% yield) was obtained as a red oil.

LCMS: MS (ESI) Retention time: 0.865 min (M+H)⁺=591.4.

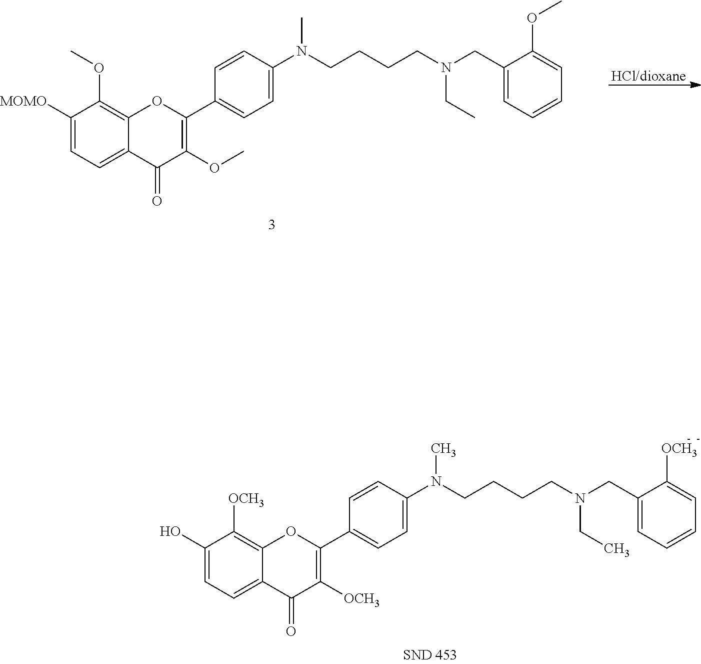

To a solution of Compound 3 (300 mg, 507.87 μmol, 1.00 eq) in HCl/dioxane (3 mL). The mixture was stirred at 25° C. for 1 hr. LCMS (EW30189 —396-P1A) showed Compound 3 was consumed completely and 76.482% of desired mass was detected. The reaction mixture was concentrated under reduced pressure to give a residue. The crude product was purified by reversed-phase HPLC (0.1% HCl condition). Compound 007-016 (SND 453) (160 mg, 274.39 μmol, 54.03% yield, 100% purity, HCl) was obtained as a yellow oil, which was determined by HNMR and LCMS.

TLC (SiO₂, PE: EA=0/1, Rf=0.4).

LCMS: MS (ESI) Retention time: 0.823 min (M+H)⁺=547.4.

LCMS: MS (ESI) Retention time: 1.438 min (M+H)⁺=547.2.

¹H NMR (400 MHz, DMSO-d₆) δ=9.87 (br s, 1H), 7.99-7.97 (d, J=9.2 Hz, 2H), 7.63-7.61 (d, J=8.8 Hz, 1H), 7.50 (m, 1H), 7.46-7.40 (m, 1H), 7.11-7.09 (d, J=8.4 Hz, 1H), 7.04-7.00 (m, 2H), 6.94-6.92 (d, J=8.8 Hz, 2H), 4.25-4.22 (t, J=4.4 Hz, 2H), 3.90 (s, 3H), 3.82 (s, 3H), 3.77 (s, 3H), 3.46 (s, 2H), 3.01 (m, 7H), 7.80-7.76 (m, 2H), 1.59-1.53 (m, 2H), 1.28-1.25 (t, J=7.2 Hz, 3H).

### Synthesis of Compound 007-017 (SND 457)

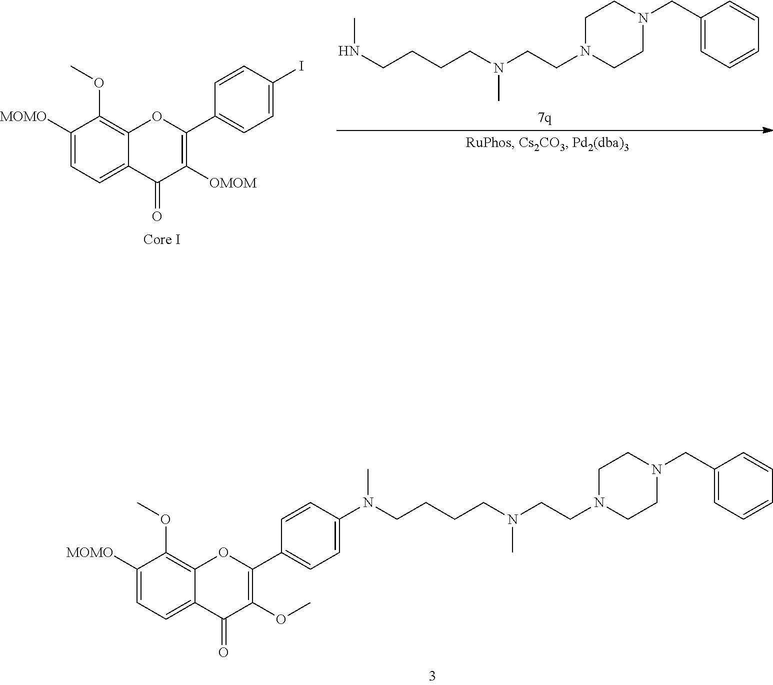

To a solution of Core J (300 mg, 640.70 μmol, 1.00 eq), RuPhos (59.80 mg, 128.14 μmol, 0.20 eq), Pd₂ (dba)₃ (117.34 mg, 128.14 μmol, 0.20 eq) and Cs₂CO₃ (626.26 mg, 1.92 mmol, 3.00 eq) in DMF (1 mL), then added Compound 7q (204.06 mg, 640.70 μmol, 1.00 eq) at 25° C., that mixture was degassed and purged with N₂ for 3 times, the mixture was stirred at 100° C. for 12 hrs under N₂ atmosphere. LCMS showed Core J consumed. Several new peaks were shown on LCMS and 38.038% of desired compound was detected. The reaction mixture was filtered and concentrated under reduced pressure to give a residue. Without purification. It was put in the next reaction. Compound 3 (300 mg, 455.36 μmol, 71.07% yield) was obtained as a red oil.

LCMS: MS (ESI) Retention time: 0.783 min (M+H)⁺=659.5.

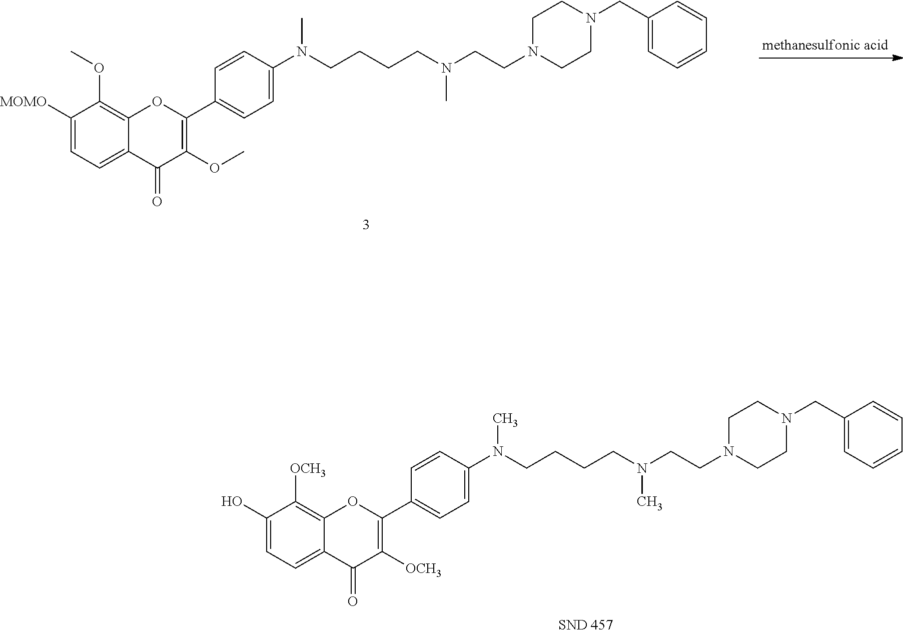

To a solution of Compound 3 (300 mg, 455.36 μmol, 1.00 eq) in HCl/dioxane (10 mL). The mixture was stirred at 25° C. for 1 hr. LCMS showed Compound 3 was consumed completely and 47.494% of desired mass was detected. The reaction mixture was concentrated under reduced pressure to give a residue. The crude product was purified by reversed-phase HPLC (0.1% HCl condition). Compound 007-017 (SND 457) (230 mg, 353.18 μmol, 77.56% yield, 100% purity, HCl) was obtained as a yellow oil, which was determined by HNMR and LCMS.

TLC (SiO₂, DCM: MeOH=10/1, Rf=0.3).

LCMS: MS (ESI) Retention time: 0.494 min (M+H)⁺=615.2.

LCMS: MS (ESI) Retention time: 1.159 min (M+H)⁺=615.2.

¹H NMR (400 MHz, DMSO-d₆) δ=11.01 (br s, 1H), 8.00-7.98 (d, J=9.2 Hz, 2H), 7.69-7.68 (m, 2H), 7.64-7.62 (d, J=8.8 Hz, 1H), 7.47-7.45 (m, 3H), 7.05-7.03 (d, J=8.8 Hz, 1H), 6.95 (br d, J=8.4 Hz, 2H), 4.43 (br s, 2H), 3.91 (s, 3H), 3.77 (s, 3H), 3.72-3.70 (m, 14H), 3.54-3.46 (m, 14H), 3.16 (m, 2H), 3.04 (s, 3H), 2.79 (s, 3H), 1.79-1.76 (m, 2H), 1.60-1.59 (m, 2H).

### Synthesis of Compound 007-020 (SND 461)

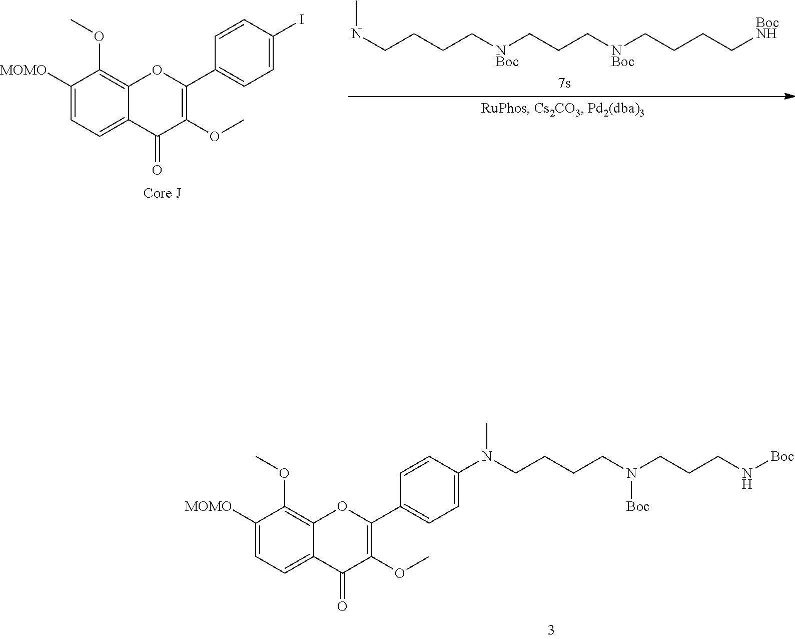

To a solution of Compound 7s (221.35 mg, 640.70 μmol, 1.00 eq) and Core J (300 mg, 640.70 μmol, 1.00 eq) in DMF (5 mL) was added RuPhos (59.80 mg, 128.14 μmol, 0.20 eq), Cs₂CO₃ (626.26 mg, 1.92 mmol, 3.00 eq) and Pd₂ (dba)₃ (117.34 mg, 128.14 μmol, 0.20 eq) at 25° C. And that mixture was degassed and purged with N₂ for 3 times. The mixture was stirred at 100° C. for 12 hrs under N₂ atmosphere. LCMS showed Core J consumed. Several new peaks were shown on LCMS and 57.282% of desired mass was detected. The reaction mixture was filtered and concentrated under reduced pressure to give a residue. Without purification. It was put in the next reaction. Compound 3 (400 mg, 583.26 μmol, 91.03% yield) was obtained as a red oil.

LCMS: MS (ESI) Retention time: 1.049 min (M+H)⁺=686.5.

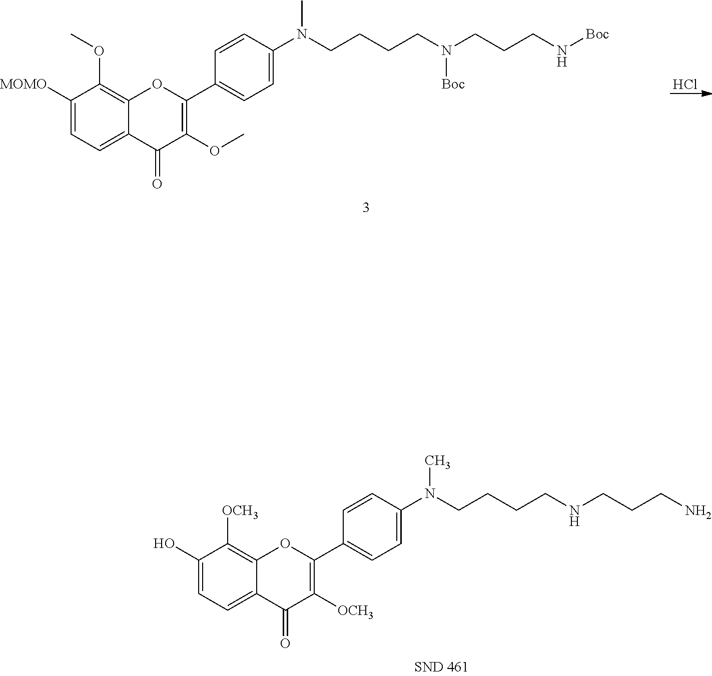

To a solution of Compound 3 (300 mg, 437.44 μmol, 1.00 eq) in HCl/dioxane (5.00 mL). The mixture was stirred at 25° C. for 1 hr. LCMS showed Compound 3 was consumed completely and 38.215% of desired mass was detected. The reaction mixture was concentrated under reduced pressure to give a residue. The crude product was purified by reversed-phase HPLC (column: 3_Phenomenex Luna C18; 75×30 mm×3 um; mobile phase: [water (HCl) -ACN]; B %: 1%-26%, 8 min). Compound 007-020 (SND 461) (110 mg, 230.14 μmol, 52.61% yield, 100% purity, HCl) was obtained as a yellow solid. Which was determined by HNMR and LCMS.

TLC (SiO₂, DCM: MeOH=10/1, Rf=0.05).

LCMS: MS (ESI) Retention time: 0.424 min (M+H)⁺=442.1.

LCMS: MS (ESI) Retention time: 0.879 min (M+H)⁺=442.1.

¹H NMR (400 MHz, DMSO-d₆) δ=9.29 (br s, 2H), 8.20 (br s, 3H), 7.93-7.91 (d, J=8.8 Hz, 2H), 7.63-7.61 (d, J=8.8 Hz, 1H), 7.03-7.01 (d, J=8.8 Hz, 1H), 6.87-6.85 (d, J=8.8 Hz, 2H), 3.90 (s, 3H), 3.76 (s, 3H), 3.19-3.15 (t, J=6.8 Hz, 2H), 2.98-2.90 (m, 6H), 2.04-1.98 (m, 2H), 1.77-1.75 m, 2H), 1.67-1.64 (m, 2H).

### Synthesis of Compound 007-021 (SND 458)

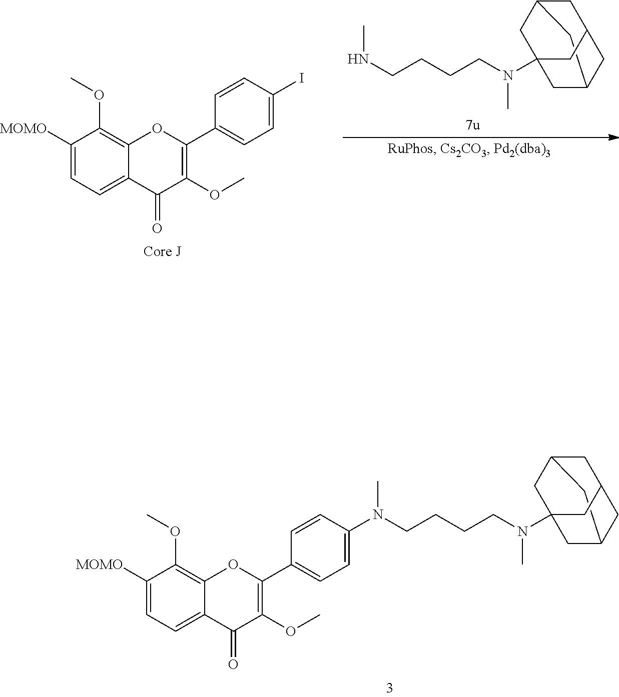

To a solution of Core J (300 mg, 660.49 μmol, 1.00 eq), RuPhos (61.64 mg, 132.10 μmol, 0.20 eq), Pd₂ (dba)₃ (120.96 mg, 132.10 μmol, 0.20 eq) and Cs₂CO₃ (645.60 mg, 1.98 mmol, 3.00 eq) in DMF (5 mL), then added Compound 7u (165.40 mg, 660.49 μmol, 1.00 eq) at 25° C., the mixture was stirred at 100° C. for 16 hrs under N₂. LCMS showed Core J was consumed. Several new peaks were shown on LCMS and 23.777% of desired compound was detected. The reaction mixture was filtered and concentrated under reduced pressure to give a residue. Without purification. It was put in the next reaction. Compound 3 (300 mg, 507.83 μmol, 76.89% yield) was obtained as a red oil.

LCMS: MS (ESI) Retention time: 0.875 min (M+H)⁺=591.4.

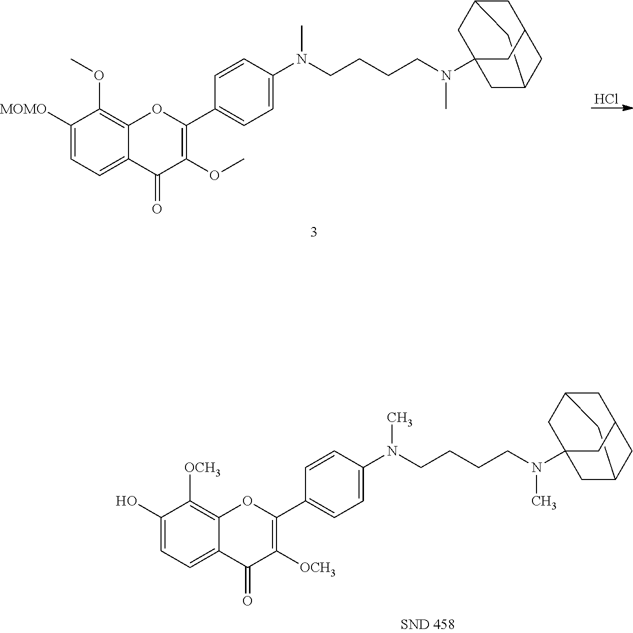

To a solution of Compound 3 (300 mg, 507.83 μmol, 1.00 eq) in HCl/dioxane (5 mL). The mixture was stirred at 25° C. for 1 hr. LCMS showed Compound 3 was consumed completely and 48.811% of desired mass was detected. The reaction mixture was concentrated under reduced pressure to give a residue. The crude product was purified by reversed-phase HPLC (0.1% HCl condition). Compound 007-021 (SND 458) (140 mg, 232.33 μmol, 45.75% yield, 96.774% purity, HCl) was obtained as a yellow oil, which was determined by HNMR and LCMS.

TLC (SiO₂, DCM: MeOH=10/1, Rf=0.05).

LCMS: MS (ESI) Retention time: 0.843 min (M+H)⁺=547.4.

LCMS: MS (ESI) Retention time: 1.795 min (M+H)⁺=547.4.

¹H NMR (400 MHz, DMSO-d₆) δ=10.06 (br s, 1H), 8.00-7.99 (d, J=7.2 Hz, 2H), 7.63-7.61 (m, 1H), 7.06-7.04 (m, 3H), 3.90 (d, J=2.4 Hz, 3H), 3.77 (d, J=2.4 Hz, 3H), 3.48 (s, 2H), 3.35 (m, 1H), 3.04-3.03 (d, J=1.6 Hz, 3H), 2.70 (m, 1H), 2.61 (s, 3H), 2.13 (s, 3H), 2.00-1.89 (m, 3H), 1.79-1.75 (m, 3H), 1.65 (m, 2H), 1.60 (m, 8H).

### Examples—Biological Studies

### Experimental Methodology

### Antitumor Activity Against a Panel of Cancer Cell Lines

Antitumor activity of the compounds and cisplatin as a positive control was assessed by using CellTiter-Glow® Luminescent Cell Viability assay (Promega #G7572) according to the manufacturer's instructions. The compounds were tested at 2 concentrations, 2 and 20 μM, in triplicate well conditions.

Tumor cells were grown at 37° C. in a humidified atmosphere with 5% CO₂ in RPMI 1640 or DMEM medium, supplemented with 10% (v/v) fetal calf serum and 50 μg/ml gentamicin for up to 20 passages, and were passaged once or twice weekly. Cells were harvested using TrypLE or PBS buffer containing 1 mM EDTA, and the percentage of viable cells is determined using a cell counter.

Cells were harvested from exponential phase cultures, counted and plated in 96 well flat-bottom microtiter plates at a cell density depending on the cell line's growth rate (4,000-20,000 cells/well depending on the cell line's growth rate, up to 60,000 for hematological cancer cell lines) in RPMI 1640 or DMEM medium supplemented with 10% (v/v) fetal calf serum and 50 μg/ml gentamicin (140 μl/well). Cultures were incubated at 37° C. and 5% CO₂ in a humidified atmosphere. After 24 h, 10 μl of test compounds or control medium are added and left on the cells for another 72 h.

Compounds were serially diluted in DMSO, transferred in cell culture medium, and added to the assay plates. The DMSO concentration was kept constant at <0.2% v/v across the assay plate. Viability of cells was quantified by CellTiter-Glow® Luminescent Cell Viability assay (Promega). Luminescence was measured with the microplate luminometer (EnVision Multi Label Reader).

Cell lines tested are presented in Table 1.

TABLE 1
Tumour cell lines type and designation

| Tissue Origin/Disease | Cell Line Name |
|---|---|
| Brain | SK-N-SH |
| | LN-229 |
| | U-118 MG |
| | SH-SY-5Y |
| Colon | HCT 116 |
| Leukemia | HL-60 |
| | K-562 |
| | U937 |
| Lung | NCI-H460 |
| | NCI-H1299 |
| | A549 |
| | NCI-H1437 |
| | NCI-H292 |
| | Calu-1 |
| | NCI-H69 |
| | DMS 114 |
| | NCI-H441 |
| Lymphoma | Daudi |
| | NAMALWA |
| Muscle | A-673 |
| Skin | A-375 |
| | MeWo |
| | SK-MEL-5 |
| | A-2058 |

### Example 1. Activity Against Brain Carcinomas

The test compounds which inhibited brain cancer cell growth >50% at a concentration of 2 μM are presented in Table 2A and 2B.

The brain carcinoma cells H118MG growth was inhibited 97.6% by SND437 at 2 μM.

TABLE 2A
% Inhibition against brain carcinoma cell lines

| Cpd/% Inh @2 μM | SK-N-SH |
|---|---|
| SND437 | 96.4 |
| SND438 | 53.5 |
| SND439 | 57.5 |

TABLE 2B
% Inhibition against brain carcinoma cell lines

| Cpd/% Inh @2 μM | SH-SY-5Y |
|---|---|
| SND446 | 55.8 |

### Example 2. Activity Against Colon Carcinoma

The test compounds which inhibited colon cancer cell growth >50% at a concentration of 2 μM are presented in Table 3.

TABLE 3
% Inhibition against colon carcinoma cell lines

| Cpd/% Inh @2 μM | HCT116 |
|---|---|
| SND437 | 83.7 |

### Example 3. Activity Against Leukaemia

The test compounds which inhibited leukemia cell growth >50% at a concentration of 2 μM are presented in Table 4. SND446 at 2 μM inhibited the growth of U937 cell line with 58%.

TABLE 4
% Inhibition against leukemia cell lines

| Cpd/% Inh @2 μM | K562 |
|---|---|
| SND437 | 65.3 |

### Example 4. Activity Against Lung Carcinomas

The test compounds which inhibited lung carcinoma cell growth >50% at a concentration of 2 μM are presented in Table 5. The lung carcinoma cells DMS114 growth was inhibited 98% by SND437 at 2 μM.

TABLE 5
% Inhibition against lung carcinoma cell lines

| Cpd/% Inh @2 μM | NCI-H292 |
|---|---|
| SND437 | 71.6 |
| SND438 | 51.9 |
| SND439 | 62.6 |

### Example 5. Activity Against Lymphoma

The test compounds which inhibited lymphoma cell growth >50% at a concentration of 2 μM are presented in Table 6.

TABLE 6
IC50 values against lymphoma

| Cpd/% Inh @2 μM | NAWALMA | Daudi |
|---|---|---|
| SND437 | 80.8 | 59.4 |
| SND438 | 65.3 | 50 |
| SND439 | 73.4 | 57.4 |
| SND446 | 75.5 | 82 |

### Example 7. Activity Against Muscle Carcinoma (Ewings Sarcoma)

The test compounds which inhibited Ewings sarcoma cell growth >50% at a concentration of 2 μM are presented in Table 7.

TABLE 7
IC50 values against sarcoma

| Cpd/% Inh @2 μM | A673 |
|---|---|
| SND437 | 99.6 |
| SND438 | 55.6 |
| SND439 | 80.8 |
| SND445 | 61.8 |
| SND459 | 63.5 |

### Example 8. Activity Against Skin Cancer (Melanoma)

The test compounds which inhibited melanoma cell growth >50% at a concentration of 2 μM are presented in Table 8. SND437 and SND446 at 2 μM inhibited the growth of MeWo and SK-Mel-5 cell line 93.2% and 56.5% respectively.

TABLE 8
IC50 values against melanoma cell lines

| Cpd/% Inh @2 μM | A2058 | A375 | SK-Mel-5 |
|---|---|---|---|
| SND437 | 97.3 | 69.5 | 99.7 |
| SND438 | 72.1 | 55.5 | 61.1 |
| SND438 | 78.2 | 60.8 | 50.7 |

It will be understood that the present invention has been described above byway of example only. The examples are not intended to limit the scope of the invention. Various modifications and embodiments can be made without departing from the scope and spirit of the invention, which is defined by the following claims only.

1. A first aspect of the invention provides a compound of formula (1): 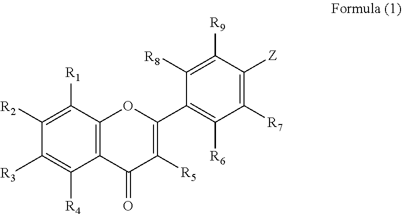
wherein:
Z is selected from:
—NR¹¹R¹²;
—N(R¹⁰)—(CH₂)ₚ—NR¹¹R¹²;
—N(R¹⁰)—(CH₂)_{q}—N(R¹⁰)—(CH₂)_{q}—NR¹¹R¹²; and
—N(R¹⁰)—(CH₂)ᵣ—N(R¹⁰)(CH₂)ᵣ—N(R¹⁰)(CH₂)ᵣ—NR¹¹R¹²;
R¹, R², and R⁴ independently, are selected from —OH, —O—C₁₋₄ alkyl, —OC(O)R₁₃, —OC(O)NHR¹³, —OC(O)N(R¹³)₂; or R¹ and R² together form —O—CH₂—O—;
R³, R⁴, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(RD)₂; —CHO; —COR^{β}; —COOH; —COOR^{β}; —OCOR^{β}; and benzyl optionally substituted with 1-3 —R^{β};
each —R^{β} is independently selected from a C₁-C₆ alkyl, C₂-C₆ alkenyl, C₂-C₆ alkynyl, —O(C₁₋₂ alkyl) or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
each R¹⁰ is independently selected from H, C₁₋₆ alkyl, C₂-C₆ alkenyl, C₂₋₆ alkynyl, C₃₋₁₀ cycloalkyl, and benzyl, wherein each R¹⁰, when not H, is independently optionally substituted with 1 or 2 —R^{β};
R¹¹ and R¹² are independently selected from H, C₁₋₆-alkyl, C₂-C₆ alkenyl, C₂₋₆ alkynyl, C₃₋₁₀ cycloalkyl, and benzyl, wherein each R¹¹ and R¹², when is not H, are independently optionally substituted with 1 or 2 —R^{β}; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl or benzyl;
each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl;
each p is independently an integer selected from 1 to 4;
each q is independently an integer selected from 1 to 4; and
each r is independently an integer selected from 1 to 4.

2. A compound as claimed in claim 1, wherein R¹, R², and R⁵, independently, are selected from —OH, and —O—C₁₋₄ alkyl.

3. A compound as claimed in claim 2, wherein R¹, R², and R⁵ are independently selected from —OH and —OCH₃.

4. A compound as claimed in any one or more of the preceding claim, wherein R³, R⁴, R⁶, R⁷, R⁸, and R⁹, independently, are selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(RD)₂; —CHO; —COR^{β}; —COOR^{β}; and benzyl optionally substituted with 1-3 —R^{β}.

5. A compound as claimed in any one or more of the preceding claim, wherein R³, R⁴, R⁶, R⁷, R⁸, and R⁹, independently, are selected from H; halo; —CN; —NO₂; —R^{β}; —NH₂; —NHR^{β}; —N(RD)₂; —CHO; —COR^{β}; —COOH; and —COOR^{β}.

6. A compound as claimed in any one or more of the preceding claim, wherein R³, R⁴, R⁶, R⁷, R⁸, and R⁹, independently, are selected from H; halo; —CN; —NO₂; and —NH₂.

7. A compound as claimed in any preceding claim, wherein R³, R⁴, R⁶, R⁷, R⁸, and R⁹, are H.

8. A compound as claimed in claim 1; wherein R¹, R² and R⁵ are independently selected from —OH and —O—C₁₋₄ alkyl, e.g. —OH and —OCH₃; and R³, R⁴, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(RD)₂; —CHO; —COR^{β}; —COOH; —COOR^{β}; —OCOR^{β}; and benzyl optionally substituted with 1-3 —R^{β}.

9. A compound as claimed in claim 1; wherein R¹, R² and R⁵, independently, are selected from —OH and —OCH₃; and R³, R⁴, R⁶, R⁷, R⁸, and R⁹, independently, are selected from H; halo; —CN; —NO₂; —SH; —SO₂H; and —NH₂.

10. A compound as claimed in any one or more of the preceding claims; wherein R¹¹ and R¹² are independently selected from H and C₁₋₆ alkyl; or R¹¹ and R¹² together form a 5- or 6-membered heterocycle optionally having an additional heteroatom selected from N and O; wherein the 5- or 6-membered heterocycle is optionally substituted with 1 or 2 C₁₋₄ alkyl or benzyl.

11. A compound as claimed in claim 11; wherein R¹¹ and R¹² together form a 5- or 6-membered heterocycle optionally substituted with 1 or 2 C₁₋₄ alkyl or benzyl.

12. A compound as claimed in claim 12; wherein the 5- or 6-membered heterocycle is morpholine, piperidine, piperazine, or pyrrolidine optionally substituted with 1 or 2 C₁₋₄ alkyl or benzyl.

13. A compound as claimed in any of claims 1 to 10, wherein R¹¹ and R¹² are independently selected from H, C₁₋₆ alkyl, and benzyl, wherein the benzyl group is optionally substituted with —OCH₃.

14. A compound as claimed in any preceding claim, wherein the compound is a compound of Formula (1A): 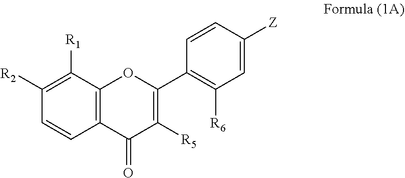
wherein R¹, R², R⁵, R⁶, and Z are as defined in claims 1 to 13.

15. A compound as claimed in claim 1, wherein the compound is selected from Table A.

16. A pharmaceutically acceptable salt, multi-salt, solvate or prodrug of a compound as defined in any one of claims 1 to 15.

17. A pharmaceutical composition comprising a compound as defined in any one of claims 1 to 15, or a pharmaceutically acceptable multi-salt, solvate or prodrug as defined in claim 16, and a pharmaceutically acceptable excipient.

18. A compound as defined in any one of claims 1 to 15, or a pharmaceutically acceptable multi-salt, solvate or prodrug as defined in claim 16, or a pharmaceutical composition as defined in claim 17, for use in medicine.

19. A compound as defined in any one of claims 1 to 15, or a pharmaceutically acceptable multi-salt, solvate or prodrug as defined in claim 16, or a pharmaceutical composition as defined in claim 17, for use treating or preventing cancer.

20. A method of treatment or prevention of a disease, disorder or condition, the method comprising the step of administering an effective amount of a compound as defined in any one of claims 1 to 15, or a pharmaceutically acceptable multi-salt, solvate or prodrug as defined in claim 16, or a pharmaceutical composition as defined in claim 19, to thereby treat or prevent the disease, disorder or condition.

21. A method of treatment as claimed in claim 17, wherein the disease, disorder or condition is cancer.
